SEQUENCE LISTING (34 sequences)

SEQ ID NO: 1 - protein, 101 aa, synthetic construct
  REGION 1..101: note = synthesized
GTFSSYAISWVRQAPGQGLEWMGGIIPIISWVRQAPGQGLEWMGGIIPIFGTANYAQKFQGRVTITADESTSTAYMELSSLRSEDTNYLAWYQQKPGVWGQ

SEQ ID NO: 2 - protein, 100 aa, synthetic construct
  REGION 1..100: note = synthesized
AIQLTQSPSSLSASASGGTNYLAWYQQKPGVRQAPGQGLEWMGGIINYLAWYQQKPGSLESGVPSKFSASGSGRDFTLIISSLQPEDFATYYCQQYNSYP

SEQ ID NO: 3 - protein, 100 aa, synthetic construct
  REGION 1..100: note = synthesized
DNYLAWYQQKPGGKAPKLLIYAAQRISTYLAWYQQKPGKVPKNYLAWYQQKPGQSGVPSRFSGTGSGTDFTLTISRLQPDDYATYYCQQYNYLAWYQQKP

SEQ ID NO: 4 - protein, 98 aa, synthetic construct
  REGION 1..98: note = synthesized
NYLAWYQQKPGFSSYAISWVRQAPGQGLEWMGGIIPIFGTANYAQNYLAWYQQKPGADESTSTAYMELSSLRSEDTAVYYCARSGSYYYGMDVWGQGT

SEQ ID NO: 5 - protein, 98 aa, synthetic construct
  REGION 1..98: note = synthesized
GKAPKLLIYAATITCRASQSINRYLNWYQQKPGKAPKLSGSGSGTDFTLTLLIYAASGSGSGTDFTLTISSLQPEDFATYYCQQYNFYPLTFGGGTKV

SEQ ID NO: 6 - protein, 99 aa, synthetic construct
  REGION 1..99: note = synthesized
SGSGSGTDFTLTTFSSYAISWVRQAPGQGLEWMGGIIPIFGSGSGSGTDFTLTTITADESTSTAYMELSSLRSEDTAVYYCARTRGYSASTLQRGVPSR

SEQ ID NO: 7 - protein, 102 aa, synthetic construct
  REGION 1..102: note = synthesized
DASTLQRGVPSRFGGSGQMTQSPSSASTLQRGVPSRFGGSGSQSINRASTLQRGVPSRFGGSGGKAPKLVIYAASSLQSGVPSRFSGSGSGTDFTLTISSLQ

SEQ ID NO: 8 - protein, 101 aa, synthetic construct
  REGION 1..101: note = synthesized
ASTLQRGVISWVRASTLQRGVPSRFGGSGPIFGTANYAQKFQGRVTITADESTSTAYMELSSLRSEDTAVYYASTLQRGVPSRFGGSGYYWGQGTPVTVSS

SEQ ID NO: 9 - protein, 98 aa, synthetic construct
  REGION 1..98: note = synthesized
SSLQSGVPSRFSGSGFGTDFTLPASTLQRGVPSRFGGSGIVIYAASSLQSGVPSRFSGSGSGTDFTLTISSLQPEDFATYYCQQYNFYPLTFGGGTKV

SEQ ID NO: 10 - protein, 100 aa, synthetic construct
  REGION 1..100: note = synthesized
SNYLNWYQSGVPSRFSGSGFGTDFTLKASGGTFSSYAISWVRQAPGQGLEWMGSNYLNWYQYAQKFQGRVTITADESTSTAYMELSSLRSEDTAVYYCAR

SEQ ID NO: 11 - protein, 101 aa, synthetic construct
  REGION 1..101: note = synthesized
SNYLNWYQSQSINRYLNWYQQKPGKAPKLVIYAASSLQYCQQYNFYPLTFGGGTKVSNYLNWYQSNYLNWYQSSGTYLNWYQQRPGKAPSSGTYQRPGKAP

SEQ ID NO: 12 - protein, 103 aa, synthetic construct
  REGION 1..103: note = synthesized
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTRGYS

SEQ ID NO: 13 - protein, 109 aa, synthetic construct
  REGION 1..109: note = synthesized
APKLLIYAASSLRSGVPSRFSGSTCRASQSINRYLNWYQAPKLLIYAASSLRSGVPSRFSGSYAASSLQSGVPSRFSGSGSGTDFTLTISSLQPEDFATYYCQQGGTKV

SEQ ID NO: 14 - protein, 111 aa, synthetic construct
  REGION 1..111: note = synthesized
YYWGQGTPVTVSKKPGSSVKVSAPKLLIYAASSLRSGVPSRFSGSGTFSSYAISWVRQAPGQGLEWMGGIIPIFGTANYAQKFQGRVTITADESTSTAYMELSSLRSEDTA

SEQ ID NO: 15 - protein, 107 aa, synthetic construct
  REGION 1..107: note = synthesized
APARFSGSGSGTEFTFTITCRASQSINRYLNWYQQKPGKAPKLVIYAASAPARFSGSGSGTEFTFTGSGSGTDFTLTISSLQPEDFATYYCQQYNFYPLTFGGGTKV

SEQ ID NO: 16 - protein, 110 aa, synthetic construct
  REGION 1..110: note = synthesized
ISSLQPDDFAAYYCQQASGGTFSSYAISWVRQAPGQGLEWMGGIIPIFGTANISSLQPDDFAAYYCQQSTSTAYMELSSLRSEDTAVYYCARTRGYSYISSLQPDDFAAY

SEQ ID NO: 17 - protein, 110 aa, synthetic construct
  REGION 1..110: note = synthesized
SLRSGVPSRFSGSGFGTNRYLNWYQQKPGKAPKLVIYAASSSLRSGVPSRFSGSGFGTDFTLTISSSLRSGVPSRFSGSGFGTLQPEDFATYYCQQYNFYPLTFGGGTKV

SEQ ID NO: 18 - protein, 109 aa, synthetic construct
  REGION 1..109: note = synthesized
GSSVKVSCKASGGTFSLRSGVPSRFSGSGFGTGQGLEWMGGIIPIFGTANYAQKFQGRVTITADESTSTAYMELSSLRSEDTAVYYCARTRGYSLRSGVPSRFSGSGFG

SEQ ID NO: 19 - protein, 103 aa, synthetic construct
  REGION 1..103: note = synthesized
DSSGTYLNWYQQRPGKAPTITCRASQSINRYLNWYQQKPGKAPKLVIYAASSLQSGVPSRFSGSGSGTDFTLTISSLQPEDFATYYCQQYNFYPLTFGGGTKV

SEQ ID NO: 20 - protein, 109 aa, synthetic construct
  REGION 1..109: note = synthesized
SSVKVSCKASGGTFSSSGTYLNWYQQRPGKAPLEWMGGIIPIFGTANYAQKFQGRVTITADESTSTAYMELSSLRSEDTAVYYCARTRGYSYGYGLYYWGQGTPVTVSS

SEQ ID NO: 21 - protein, 94 aa, synthetic construct
  REGION 1..94: note = synthesized
APGQGLEPGVRQAPGQGTFSSYAISWVRQAPGQGWMGGIIPIFGTANYAQKFQGRVTITADESTSTAYMELSSLRSEDTNYLAWYQQKPGVWGQ

SEQ ID NO: 22 - protein, 117 aa, synthetic construct
  REGION 1..117: note = synthesized
AIQLTQSPSSLSADNYLAWYQQKPGGKAPKLLIYSASGGTNYLGIIPIISWVRQGLEWMGGIINYLAWYQQKPGSLESGVPSKFSASGSGRDFTLIISSLQPEDFATYYCQQYNSYP

SEQ ID NO: 23 - protein, 99 aa, synthetic construct
  REGION 1..99: note = synthesized
TLQRGVPSRFGGSGPIFGTANYAQKFQGRLLIYAAGKVPKNYLAWYQQKPGQSGVPSRFSGTGSGTDFTLTISRLWMGGIIPIFGTANYAQNYQPDQKP

SEQ ID NO: 24 - protein, 98 aa, synthetic construct
  REGION 1..98: note = synthesized
NYLAQRISTAWYQQKLEWMGYLAWYQQKPWYQQKPGFSSYAISWVRQAPGQGLELAWDYATYYCQQYNYLAWYQYQQKPGADESTSTAYMELSSLRSE

SEQ ID NO: 25 - protein, 112 aa, synthetic construct
  REGION 1..112: note = synthesized
DNYLAWYDTAVYYCARSGSYYYGMDVWGQGTQQKPGGKAPKLLIYAAQRISTAWYQQKLEWMGYLAWYQQKPGKVPKNYLAWYQQKPGQSGVPSRFSGTGSGTDFTLTISRL

SEQ ID NO: 26 - protein, 108 aa, synthetic construct
  REGION 1..108: note = synthesized
WMGGIIPIFGTANYAQNYQPDQKPNYLAWYQQKPGFSSYAISWVRQAPGQGLELAWDYATYYCQQYNYLAWYQYQQKPGADESTSTAYMELSSLRSEDTAVYYCARSG

SEQ ID NO: 27 - protein, 112 aa, synthetic construct
  REGION 1..112: note = synthesized
VYYCARSGSYYYGMDVDNYLAWYDTAWGQGTQQKPGGKAPKLLIYAAWYQQKPGKVPKNYLAWYQQKPGQSGVPSRFSGTGSGTDFTLTISRLAQRISTAWYQQKLEWMGYL

SEQ ID NO: 28 - protein, 108 aa, synthetic construct
  REGION 1..108: note = synthesized
WYQQKPGFSSYAISWVRQAWMGGIIPIFGTANYAQNYQPDQKPNYLAPGQGLELAWDYATYQYQQKPGADESTSTAYMELSSLRSEDTYYCQQYNYLAWAVYYCARSG

SEQ ID NO: 29 - protein, 101 aa, synthetic construct
  REGION 1..101: note = synthesized
YQSQSINRYLNWYQQKPGKSNYLNWAPKLVIYAASSLQYCQQYNFYPLTFGYLNWYQSSGTYLNWYQQRPGKAPSSGTYQRPGKAPGGTKVSNYLNWYQSN

SEQ ID NO: 30 - protein, 103 aa, synthetic construct
  REGION 1..103: note = synthesized
YAQKFQGRVTITVKVSCKASGGTFSSYAISWVRQAPGQGLEWMGQVQLVQSGAEVKKPGSSGIIPIFGTANADESTSTAYMELSSLRSEDTAVYYCARTRGYS

SEQ ID NO: 31 - protein, 101 aa, synthetic construct
  REGION 1..101: note = synthesized
QRGVPSRFGGSGIVIYAASYQSQSINRYLNWYAASSLQYCQQYNFYPLTFGYLNWYQSSGTYLNWYQQRPGKAPSSGTYQRPGKAPGGTKVSNYLNWYQSN

SEQ ID NO: 32 - protein, 102 aa, synthetic construct
  REGION 1..102: note = synthesized
QQKPGKSNYLNWAPKLVIYYAQKFQGRVTITVKVSCKASGGTFQGLEWMGQVQLVQSPIFGTANADESTSTAYMELSSLRSEDTAVYYCARTRGYSGAEVKK

SEQ ID NO: 33 - protein, 101 aa, synthetic construct
  REGION 1..101: note = synthesized
NWYAASSLQYCQQYNFYPLTFKAPGGTKVSNYLNWYQSNGYLNWYQSSGTYLNWYQQRPGKAPSSGTYQRPGQRGVPSRFGGSGIVIYAASYQSQSINRYL

SEQ ID NO: 34 - protein, 102 aa, synthetic construct
  REGION 1..102: note = synthesized
ESTSTAYMELSSLRFQGRVTITVKVSCKASGGTFQGLEWMGQVQLVQSPIFGTANADSEDTAVYYCARTRGYSGAEVKKQQKPGKSNYLNWAPKLVIYYAQK

(19) United States
(12) Patent Application Publication
(10) Pub. No.: US 2023/0382984 A1
(43) Pub. Date: Nov. 30, 2023

(54) TRABD PROTEIN, AND MONOCLONAL ANTIBODY THEREOF, AND ISOLATION, AMPLIFICATION AND USE THEREOF

(71) Applicant: Xiao GUAN, Zhenjiang (CN)

(72) Inventor: Xiao GUAN, Zhenjiang (CN)

(21) Appl. No.: 18/322,110

(22) Filed: May 23, 2023

Related U.S. Application Data
(60) Provisional application No. 63/365,274, filed on May 25, 2022.

(30) Foreign Application Priority Data
May 25, 2022 (CN) 202210580785.9
May 25, 2022 (CN) 202210580786.3
May 25, 2022 (CN) 202210583677.7

(51) Int. Cl. C07K 16/18; C12N 15/85; A61P 35/00

(52) U.S. Cl. CPC C07K 16/18; C12N 15/85; A61P 35/00; C12N 2800/107; A61K 2039/505

(57) ABSTRACT

An anti-TRABD monoclonal antibody and use thereof are provided. By establishing a culture system of monocyte-induced dendritic cells and killer CD8⁺ T cells, it can be verified whether TRABD2A, TRABD2B or TRABD2A and TRABD2B expression is present on the surface of CD8⁺ T cell membrane, and the cells have enhanced killing effect against target cells after TRABD2A, TRABD2B or TRABD2A and TRABD2B is inhibited, so that the TRABD2A, TRABD2B or TRABD2A and TRABD2B protein can be used as an immune checkpoint. Meanwhile, by obtaining an anti-TRABD2A, TRABD2B or TRABD2A and TRABD2B monoclonal antibody, it can be proven that anti-TRABD2A monoclonal antibody, anti-TRABD2B monoclonal antibody or anti-TRABD2A and TRABD2B monoclonal antibody can improve the killing effect of CD8⁺ T cells on target tumor cells, and thus can be used as an immune checkpoint inhibitor.

### CROSS-REFERENCE TO RELATED APPLICATIONS

This application claims the priority of the previous application submitted to the State Intellectual Property Office of China on May 25, 2022, with the patent application number of CN 2022105807863 and title of “TRABD2A protein, and monoclonal antibody thereof, and isolation, amplification, application submitted to the State Intellectual Property Office of China on May 25, 2022 with the patent application number of CN 2022105807859 and the title of “TRABD2B protein, and monoclonal antibody thereof, and its isolation, amplification and application”, and application submitted to the State Intellectual Property Office of China on May 25, 2022 with the patent application number of CN 2022105836777 and the title of “TRABD protein, and bispecific antibody thereof, and isolation, amplification and application”, and provisional application submitted to United States Patent and Trademark Office on May 25, 2022 with the application No. 63/365,274 and the title of “TRABD PROTEIN, AND MONOCLONAL ANTIBODY THEREOF, AND ISOLATION, AMPLIFICATION AND USE THEREOF”. The full text of the earlier applications are incorporated herein by reference.

### INCORPORATION OF SEQUENCE LISTING

This application contains a sequence listing submitted in Computer Readable Form (CRF). The CFR file containing the sequence listing entitled “PA150-0164-SequenceListing.xml”, which was created on May 23, 2023, and is 46,390 bytes in size. The information in the sequence listing is incorporated herein by reference in its entirety.

### TECHNICAL FIELD

The present disclosure relates to TRABD2A protein, and a monoclonal antibody thereof, and isolation, amplification and use thereof, and belongs to the fields of bioengineering and biotherapy.

The present disclosure relates to TRABD2B protein, and a monoclonal antibody thereof, and isolation, amplification and use thereof, and belongs to the fields of bioengineering and biotherapy.

The present disclosure relates to TRABD protein, and a monoclonal antibody thereof, and isolation, amplification and use thereof, and belongs to the fields of bioengineering and biotherapy.

### BACKGROUND

Adoptive cell therapy (ACT) is a promising option for the treatment of cancer, and the adoptive cell therapy using antigen-specific CD8⁺ T cells means that a large number of CD8⁺ T cells from a patient, after the in vitro induction of differentiation, modification and amplification, acquire specificity for tumor cells, and then are re-infused to the patient, thereby enhancing the killing ability of CD8⁺ T cells against tumor cells. Initially, the cells used in ACT are derived from the patient's own tumor-infiltrating lymphocytes, but this ACT scheme is limited by the small number of tumor-infiltrating lymphocytes and the inability to radically enhance or improve killing ability against tumors.

Immune checkpoints are a series of molecules capable of modulating the degree of immune activation, the presence of which provides support for the immune system to prevent damaging of self-tissue cells due to excessive autoimmunity during the course of tumor immune response, and to maintain immune tolerance. However, some smart tumor cells can take advantage of this mechanism and transmit negative signals to immune cells expressing immune checkpoints, and this is a means for the tumor cells to escape immune responses. The immune checkpoint blockade therapy is directed at this phenomenon, and in this therapy, an immune checkpoint inhibitor is used to relieve inhibition and recover cytotoxicity of immune cells such as CD8⁺ T cells and the like against tumor cells.

For the research on immune checkpoints, excellent achievements have been made endlessly in recent years. Immune checkpoints PD-1, CTLA-4, TIGIT, LAG-3 and TIM3 play outstanding roles in the immune system, and over the more than ten years that have passed, many breakthroughs have been made in the research on monoclonal antibody inhibitors targeting these immune checkpoints. However, more and more researches have shown that the use of immune checkpoint blockade therapy alone is effective in a small number of patients, while it can not achieve objective effects in the majority of patients and even results in adverse effects. This is related to the type of disease and individual difference of patients. Therefore, what is now advocated is the evaluation of the effect of combined use of different immune checkpoint blockers or use of an immune checkpoint blocker in combination with other drugs and therapies having synergistic effects with the immune checkpoint blocker, thus improving the efficacy of a single drug and achieving therapeutic benefit of 1+1>2.

According to the report in American Association for Cancer Research (AACR) Annual Meeting 2019, researchers treated pancreatic cancer patients with APX005M (anti-CD40 mAb), gemcitabine (DNA synthesis inhibitor) and chemotherapeutic drugs nab-paclitaxel (NP) in combination with nivolumab (anti-PD-1 mAb), and the phase Ib clinical trial showed positive results. Recent researches have shown that treatment of patients with unresectable liver cancer with atezolizumab (anti-PD-L1 mAb) in combination with bevacizumab (anti-VEGF) has a better therapeutic response than monotherapy. This combination regimen has been approved by the FDA. Nevertheless, some patients still show different degrees of adverse reactions, and more clinical designs and data are needed to support the determination of the optimal combination regimen for different diseases.

TRABD2A and its paralog TRABD2B encode a type I transmembrane glycoprotein distributed predominantly in the cell membrane, with the structure being single-spanning and most of the structure being exposed outside the cell membrane, and the TRABD protein may function whether located inside or outside the membrane. In addition, TRABD protein contains a TraB domain and is a metalloprotease, and its activity depends on a divalent metal ion that serves as a cofactor, and thus the function of TRABD protein can be inhibited by a divalent metal ion complexing agent (EDTA or 1,10-phenanthroline). An early study showed that Co²⁺ and Mn²⁺ can increase TRABD protease activity, whereas Ni²⁺ and Cu²⁺ had opposite effect on TRABD2A protease activity. The TRABD protease lacks the zinc-binding motif (HEXXH), so the TRABD protease is also not a Zn²⁺-dependent protease. However, there is a different view that the activity of the TRABD2A protein is inhibited by Co²⁺ and Ni²⁺ and is independent of Mg²⁺, Ca²⁺ and Fe³⁺, and this may be related to different functions of the proteases.

TRABD protease has long been considered to have proteolytic enzyme activity, and it acts as a Wnt inhibitor and can cleave Wnt3a, Wnt5a and Wnt8 to achieve a deactivation effect in Wnt-secreting donor cells and Wnt acceptor cells. It is worth mentioning that, a research in 2019 reported for the first time that TRABD2A is expressed on the surface of T cell membrane, and TRABD2A is an important restriction element in the formation of human immunodeficiency virus type 1 (HIV-1). HIV-1 virus invades into a host to replicate, and assembles to form virus particles on cell membrane in later period. TRABD2A protease degrades the precursor protein of virus structural protein Gag, and thus generation and release of HIV-1 virus in CD4⁺ T cells are inhibited. The group also found that TRABD2A protease is expressed on the membranes of primary monocyte derived dendritic cells (MDDCs), and verified that TRABD2A protease also inhibits the generation and release of HIV-1 in MDDCs. CD8⁺ T cells, as an important part of adaptive immunity, play a critical role in the clearance of various pathogenic microorganisms and tumor cells. Activated killer CD8⁺ T cells, also known as cytotoxic T cells (CTLs), function primarily through three pathways: (1) production of cytokines TNF-α and IFN-γ; (2) binding of the surface FasL to Fas on the surface of a target cell to start an apoptosis signal in the target cell; (3) releasing cytotoxic particles such as perforin and granzyme to lyse the target cells. Before being secreted, the cytotoxic particle of CD8⁺ T cells need to move to an immune synapse along a microtubule after modification at the N-terminal, and then, at the interface where the CD8⁺ T cell plasma membrane and the target cell plasma membrane interacts, be butted with an endosomal “extracellular vesicle” under the action of Rab27a to form a “cytotoxic particle-extracellular vesicle” fusion vesicle, and thereby the cytotoxic particle can obtain the secretion capacity and then be secreted to the synaptic cleft to play a role. Besides the three classical killing pathways of CTL, researches show that the extracellular vesicle derived from the CD8⁺ T cells contains CD8⁺ T cell surface membrane molecules (CD3 and TCR), granzymes, cytokines and other toxic substances, and thus has a certain killing effect on target cells.

However, to date, there have been no reports of TRABD2A, TRABD2B and TRABD being associated with cellular immunity, let alone reports of TRABD2A, TRABD2B and TRABD being used in immunotherapy.

### SUMMARY

In view of the above technical problems, the present disclosure provides, in one aspect, a monoclonal antibody, which comprises: a heavy chain amino acid sequence and a light chain amino acid sequence set forth in SEQ ID NOs: 1 and 2, respectively; a heavy chain amino acid sequence and a light chain amino acid sequence set forth in SEQ ID NOs: 3 and 4, respectively; a heavy chain amino acid sequence and a light chain amino acid sequence set forth in SEQ ID NOs: 5 and 6, respectively; a heavy chain amino acid sequence and a light chain amino acid sequence set forth in SEQ ID NOs: 7 and 8, respectively; a heavy chain amino acid sequence and a light chain amino acid sequence set forth in SEQ ID NOs: 9 and 10, respectively; a heavy chain amino acid sequence and a light chain amino acid sequence set forth in SEQ ID NOs: 11 and 12, respectively; a heavy chain amino acid sequence and a light chain amino acid sequence set forth in SEQ ID NOs: 13 and 14, respectively; a heavy chain amino acid sequence and a light chain amino acid sequence set forth in SEQ ID NOs: 15 and 16, respectively; a heavy chain amino acid sequence and a light chain amino acid sequence set forth in SEQ ID NOs: 17 and 18, respectively; or a heavy chain amino acid sequence and a light chain amino acid sequence set forth in SEQ ID NOs: 19 and 20, respectively.

In a preferred embodiment of the present disclosure, the monoclonal antibody comprises: the heavy chain amino acid sequence and the light chain amino acid sequence set forth in SEQ ID NOs: 7 and 8, respectively; or the heavy chain amino acid sequence and the light chain amino acid sequence set forth in SEQ ID NOs: 13 and 14, respectively.

In another aspect, the present disclosure provides a monoclonal antibody, which comprises: a heavy chain amino acid sequence and a light chain amino acid sequence set forth in SEQ ID NOs: 21 and 22, respectively; a heavy chain amino acid sequence and a light chain amino acid sequence set forth in SEQ ID NOs: 23 and 24, respectively; a heavy chain amino acid sequence and a light chain amino acid sequence set forth in SEQ ID NOs: 25 and 26, respectively; a heavy chain amino acid sequence and a light chain amino acid sequence set forth in SEQ ID NOs: 27 and 28, respectively.

In a preferred embodiment of the present disclosure, the monoclonal antibody comprises: the heavy chain amino acid sequence and the light chain amino acid sequence set forth in SEQ ID NOs: 23 and 24, respectively; or the heavy chain amino acid sequence and the light chain amino acid sequence set forth in SEQ ID NOs: 27 and 28, respectively.

In another aspect, the present disclosure provides a monoclonal antibody, which comprises: a heavy chain amino acid sequence and a light chain amino acid sequence set forth in SEQ ID NOs: 29 and 30, respectively; a heavy chain amino acid sequence and a light chain amino acid sequence set forth in SEQ ID NOs: 31 and 32, respectively; a heavy chain amino acid sequence and a light chain amino acid sequence set forth in SEQ ID NOs: 33 and 34.

In a preferred embodiment of the present disclosure, the monoclonal antibody comprises: the heavy chain amino acid sequence and the light chain amino acid sequence set forth in SEQ ID NOs: 29 and 30, respectively.

In another aspect, the present disclosure provides a nucleotide sequence encoding the heavy chain amino acid sequence.

In another aspect, the present disclosure provides a nucleotide sequence encoding the light chain amino acid sequence.

In another aspect, the present disclosure provides a vector comprising the nucleotide sequence.

In another aspect, the present disclosure provides a host cell comprising the nucleotide sequence or the vector.

In another aspect, the present disclosure provides a method for preparing the monoclonal antibody, which comprises the steps of culturing the host cell under suitable conditions and isolating the monoclonal antibody from a cell culture.

In another aspect, the present disclosure provides use of a TRABD2A protein as an immune checkpoint.

In another aspect, the present disclosure provides use of a TRABD2B protein as an immune checkpoint.

In another aspect, the present disclosure provides use of a TRABD2A protein and a TRABD2B protein as immune checkpoints.

In another aspect, the present disclosure provides use of the TRABD2A monoclonal antibody as an immune checkpoint inhibitor.

In another aspect, the present disclosure provides use of the TRABD2B monoclonal antibody as an immune checkpoint inhibitor.

In another aspect, the present disclosure provides use of the TRABD2A and TRABD2B bispecific antibody as an immune checkpoint inhibitor.

In a preferred embodiment of the present disclosure, the TRABD2A protein or the monoclonal antibody is used in the preparation of a tumor detection reagent or an anti-tumor drug.

In another aspect, the present disclosure provides a TRABD2A protein-loaded MDDC cell.

In another aspect, the present disclosure provides a method for preparing the TRABD2A protein-loaded MDDC cell, which comprises the step of contacting a MDDC cell with TRABD2A protein.

In another aspect, the present disclosure provides a TRABD2A protein-blocked T cell.

In yet another aspect, the present disclosure provides a method for preparing the TRABD2A protein-blocked T cell, which comprises the step of contacting a T cell with TRABD2A monoclonal antibody.

In a preferred embodiment of the present disclosure, the TRABD2B protein or the monoclonal antibody is used in the preparation of a tumor detection reagent or an anti-tumor drug.

In another aspect, the present disclosure provides a TRABD2B protein-loaded MDDC cell.

In another aspect, the present disclosure provides a method for preparing the TRABD2B protein-loaded MDDC cell, which comprises the step of contacting an MDDC cell with TRABD2B protein.

In another aspect, the present disclosure provides a TRABD2B protein-blocked T cell.

In yet another aspect, the present disclosure provides a method for preparing the TRABD2B protein-blocked T cell, which comprises the step of contacting a T cell with TRABD2B monoclonal antibody.

In a preferred embodiment of the present disclosure, the TRABD2A and TRABD2B proteins or the monoclonal antibody is used in the preparation of a tumor detection reagent or an anti-tumor drug.

In another aspect, the present disclosure provides a TRABD2A and TRABD2B protein-loaded MDDC cell.

In another aspect, the present disclosure provides a method for preparing the TRABD2A and TRABD2B protein-loaded MDDC cell, which comprises the step of contacting an MDDC cell with TRABD2A and TRABD2B proteins.

In another aspect, the present disclosure provides a TRABD2A and TRABD2B protein-blocked T cell.

In yet another aspect, the present disclosure provides a method for preparing the TRABD2A and TRABD2B protein-blocked T cell, which comprises the step of contacting a T cell with TRABD2A and TRABD2B bispecific antibody.

Beneficial Effects

Compared with the prior art, the anti-TRABD2A monoclonal antibody provided herein can block TRABD2A and thus can improve the effect of killing target tumor cells by CD8⁺ T cells. The advantages mainly include the following aspects:
1. The present disclosure successfully establishes a culture system of monocyte-induced dendritic cells and killer CD8⁺ T cells, verifies that TRABD2A expression is present on the surface of CD8⁺ T cell membrane, verifies that the cells have enhanced killing effect against target cells after TRABD2A is inhibited, and reveals that TRABD2A plays a role in the process of killing the target cells by the CD8⁺ T cells, so that the TRABD protein can be used as an immune checkpoint.
2. The present disclosure successfully obtains the monoclonal antibody against TRABD antigen, and proves that the TRABD2A antibody can improve the killing effect of CD8⁺ T cells on target tumor cells. TRABD2A-blocking experiments demonstrate that the reason for the enhanced cytotoxic effect of CD8⁺ T cells may be an increased release of perforin and extracellular vesicles, and thus anti-TRABD monoclonal antibodies can be used as immune checkpoint inhibitors. Compared with the prior art, the anti-TRABD2B monoclonal antibody provided herein can block TRABD2B and thus can improve the killing effect of CD8⁺ T cells on target tumor cells. The advantages mainly include the following aspects:
1. The present disclosure successfully establishes a culture system of monocyte-induced dendritic cells and killer CD8⁺ T cells, verifies that TRABD2B expression is present on the surface of CD8⁺ T cell membrane, verifies that the cells have an enhanced killing effect against target cells after TRABD2B is inhibited, and reveals that TRABD2B plays a role in the process of killing the target cells by the CD8⁺ T cells, so that the TRABD2B protein can be used as an immune checkpoint.
2. The present disclosure successfully obtains the monoclonal antibody against TRABD2B antigen, and proves that the TRABD2B antibody can improve the killing effect of CD8⁺ T cells on target tumor cells. TRABD2B-blocking experiments demonstrate that the reason for the enhanced cytotoxic effect of CD8⁺ T cells may be an increased release of perforin and extracellular vesicles, and thus anti-TRABD2B monoclonal antibodies can be used as immune checkpoint inhibitors.

Compared with the prior art, the anti-TRABD2A and TRABD2B bispecific antibody provided herein can block both TRABD2A and TRABD2B proteins, and thus can significantly improve the effect of killing target tumor cells by CD8⁺ T cells. The advantages mainly include the following aspects:
1. The present disclosure successfully establishes a culture system of monocyte-induced dendritic cells and killer CD8⁺ T cells, verifies that TRABD2A and TRABD2B expression is present on the surface of CD8⁺ T cell membrane, verifies that the cells have significantly enhanced killing effect against target cells after TRABD2A and TRABD2B are inhibited, and reveals that TRABD2A and TRABD2B play roles in the process of killing the target cells by the CD8⁺ T cells, so that the TRABD2A and TRABD2B proteins can be used as immune checkpoints.
2. The present disclosure successfully obtains the bispecific antibody against both TRABD2A and TRABD2B antigens, and proves that the bispecific antibody can significantly improve the killing effect of CD8⁺ T cells on target tumor cells. TRABD2A and TRABD2B-blocking experiments demonstrate that the reason for the enhanced cytotoxic effect of CD8⁺ T cells may be an increased release of perforin and extracellular vesicles, and thus anti-TRABD2A and TRABD2B bispecific antibodies can be used as immune checkpoint inhibitors.

### BRIEF DESCRIPTION OF THE DRAWINGS

FIG. 1 shows induction expression of monocyte-induced dendritic cells;
A: the extraction efficiency of PBMCs, where the survival rate of a single monocyte after extraction is 99.4%, and the survival rate of the lymphocytes after extraction is 86.77%;
B: maturation of DCs under light microscopy (400×);
a: immature DCs on day 4;
b: immature DCs on day 6;
c: DCs on day 10 before being induced to be mature;
d: mature DCs on day 12;
C: identification of surface markers (CD14, CD11c, CD83 and CD86) of mature DCs.

FIG. 2 shows isolation and functional assay of killer CD8⁺ T cells;
A: antigen-loaded MDDCs being co-cultured with CD8⁺ T cells under light microscopy (100×);
B: sorting the killer CD8⁺ T cells out from the mixture of the MDDCs and the CD8⁺ T cells by using magnetic beads, where the sorting efficiency can reach 97.69%;
C: CSFE detection of CD8⁺ T cell proliferation;
D: ELISA detection of the release levels of cytokines TNF-α and IFN-7 of CD8⁺ T cell;
E: flow cytometry detection of the levels of cytokines TNF-α and IFN-7 in CD8⁺ T cells;
F: detection of the killing activity of CD8⁺ T cells on target cells (CEM.T2) under different effector-to-target ratios.

FIG. 3A shows isolation and functional assay of killer CD8⁺ T cells;
A: verification of the TRABD2A expression in CD8⁺ T cells by RNA sequencing;
B: qRT-PCR detection of the TRABD2A expression in different cells;
C: verification of the TRABD2A expression on the cell membrane of CD8⁺ T cells by immunofluorescence;
D: flow cytometry detection of the TRABD2A expression on the cell membrane of CD8⁺ T cells.

FIG. 3B shows isolation and functional assay of killer CD8⁺ T cells;
A: qRT-PCR detection of TRABD2A expression in different cells;
B: verification of the TRABD2A expression on the cell membrane of CD8⁺ T cells by immunofluorescence;
C: flow cytometry detection of the TRABD2A expression on the cell membrane of CD8⁺ T cells.

FIG. 3C shows isolation and functional assay of killer CD8⁺ T cells;
A: qRT-PCR detection of TRABD2B expression in different cells;
B: verification of the TRABD2B expression on the cell membrane of CD8⁺ T cells by immunofluorescence;
C: flow cytometry detection of the TRABD2B expression on the cell membrane of CD8⁺ T cells.

FIG. 4A show the effect on the target cell killing ability of CD8⁺ T cells after metal cofactor intervention of TRABD2A;
A: detection of the killing activity of the CD8⁺ T cells on target cells CEM.T2 when metal cations Co²⁺, Ni²⁺ and Mn²⁺ at different concentrations act on CD8⁺ T cells;
B: detection of the killing activity of the CD8⁺ T cells on target cells MGC when metal cations Co²⁺, Ni²⁺ and Mn²⁺ at different concentrations act on CD8⁺ T cells;
C: CCK8 and flow cytometry detection of the toxic effect of metal cations Co²⁺, Ni²⁺ and Mn²⁺ at different concentrations on target cells.

FIG. 4B shows the effect on the target cell killing ability of CD8⁺ T cells after metal cofactor intervention with TRABD2B;
A: in cases where different concentrations of metal cation Ni²⁺ act upon CD8⁺ T cells, detection of the killing activity of CD8⁺ T cells against target cells CEM.T2;
B: CCK8 and flow cytometry detection of the toxic effects of different concentrations of metal cation Ni²⁺ on target cells.

FIG. 5A shows the results of affinity experiment for the TRABD2A protein and antibodies;
A: graph showing experimental results for negative antibody, positive antibody-1 and positive antibody-2;
B: experimental results for monoclonal antibody Ab1-Ab10.

FIG. 5B shows the results of the affinity experiment for the TRABD2B protein and monoclonal antibodies Ab1 to Ab4.

FIG. 5C shows the results of the affinity experiment for the TRABD2A and TRABD2B proteins and bispecific antibodies Ab1 to Ab3.

FIG. 6A shows the quality detection of the TRABD2A antibodies after synthesis;
A: non-reduced SDS-PAGE analysis of synthetic antibodies purified by Mabselect Sure;
B: final SDS-PAGE results of anti-TRABD2A-1, anti-TRABD2A-2 and negative control antibodies;
C: binding curves of antibodies to antigen.

FIG. 6B shows the quality detection of the TRABD2B antibody after synthesis;
A: non-reduced SDS-PAGE analysis of synthetic antibodies purified by Mabselect Sure;
B: binding curves of anti-TRABD2B and negative control antibody to antigens.

FIG. 6C shows the quality detection of the bispecific antibody anti-TRABD2A/2B antibody after synthesis;
A: non-reduced SDS-PAGE analysis of synthetic antibodies purified by Mabselect Sure;
B. binding curves of anti-TRABD2A/2B and negative control antibody to the antigen TRABD2A protein;
C. binding curves of anti-TRABD2A/2B and negative control antibody to the antigen TRABD2B protein.

FIG. 7A shows the killing effect of CD8⁺ T cells on target cells after intervention with TRABD2A antibody;
A: killing the target cells by the CD8⁺ T cells with antibody unremoved after incubation with drugs at different concentrations;
B: killing the target cells by the CD8⁺ T cells with antibody removed after incubation with drugs at different concentrations.

FIG. 7B shows the killing effect of CD8⁺ T cells on target cells under intervention with TRABD2B antibody;
A: killing of the target cells by CD8⁺ T cells with the antibody removed after incubation with drugs at different concentrations;
B: killing of the target cells by CD8⁺ T cells with the control antibody removed after incubation with drugs at different concentrations.

FIG. 7C shows the killing effect of CD8⁺ T cells on target cells under intervention with the bispecific antibody anti-TRABD2A/2B antibody;
A: killing of the target cells by CD8⁺ T cells with the antibody removed after incubation with the antibody at different concentrations;
B: killing of the target cells by CD8⁺ T cells with the control antibody removed after incubation with the antibody at different concentrations;
C: detection of the toxic effects of different concentrations of metal cation Ni²⁺ on the target cells by using CCK8 and a flow cytometer.

FIG. 8 shows the quality evaluation of sEVs from CD8⁺ T cells after intervention with TRABD2A antibody;
A: NTA detection of particle size and concentration of sEVs collected after intervention with TRABD2A antibody and negative control antibody;
B: concentration comparison of sEVs collected after intervention with TRABD2A antibody and negative control antibody;
C: electron micrographs of sEVs collected after intervention with TRABD2A antibody and negative control antibody;
D: expression of CD8⁺ T cells and collected sEV protein after intervention with TRABD2A antibody and negative control antibody;
E: CCK8 detection of the survival of MGC803 cells under the action of sEVs collected after intervention with TRABD2A antibody and negative control antibody;
F: examination, by Transwell cell migration experiment, cell invasion experiment and plate colony formation experiment, of effect of sEVs derived from CD8⁺ T cells on the function of the tumor cells MGC803 before and after intervention with TRABD2A antibody.

FIG. 9 shows the effect of TRABD2A protein on functions of CD8⁺ T cells;
A: detection of IFN-γ and TNF-α in CD8⁺ T cells;
B: functional detection of CD8⁺ T cell protein level after antibody blocking;
C: detection, by immunofluorescence, of the positioning and expression of perforin in CD8⁺ T cells under intervention with TRABD2A antibody;
D-E: expression of TRABD2A and related genes after the TRABD2A protein gene of CD8⁺ T cells was knocked out by siRNA;
F: expression of protein after TRABD2A of CD8⁺ T cells was knocked out by siRNA.

FIG. 10 shows the effect on solid tumor in vivo versus treatment dose of killer CD8⁺ T cells;
A: change in body weight of nude mice after treatment relative to day 0;
B: change in tumor volume of nude mice relative to day 0;
C: flow cytometry detection, after treatment, of the distribution of CD8⁺ T cells in organs of nude mice treated at a dose of 2×10⁶.

FIG. 11A shows the therapeutic effect of killer CD8⁺ T cells on solid tumor in vivo under intervention with TRABD2A antibody;
A: change in body weight of nude mice after treatment relative to the day of tumor bearing;
B: change in tumor volume of nude mice relative to 4 days before treatment;
C: survival curve analysis.

FIG. 11B shows the therapeutic effect of killer CD8⁺ T cells on solid tumors in vivo under intervention with TRABD2B antibody;
A: changes in body weight of nude mice after treatment relative to the day of tumor bearing;
B: changes in tumor volume of nude mice relative to 4 days before treatment.

FIG. 11C shows the therapeutic effect of killer CD8⁺ T cells on solid tumors in vivo under intervention with the bispecific antibody TRABD2A/2B antibody;
A: changes in body weight of nude mice after treatment relative to the day of tumor bearing;
B: changes in tumor volume of nude mice relative to 4 days before treatment.

### DETAILED DESCRIPTION

For better illustrating the objectives and advantages of the present method, the detailed description of the present disclosure will be further described in detail with reference to the accompanying drawings and examples.

### Example 1: Culture and Identification of Peripheral Blood Monocyte-Induced Dendritic Cells (MDDCs)

Monocytes were isolated and cultured from peripheral blood of a healthy person, and then gradually differentiated into immature dendritic cells (imDCs) under the action of GM-CSF and IL-4. Then TRABD antigen peptide or tumor cell lysate antigen was added, and the immature dendritic cells were induced into antigen-loaded mature dendritic cells (mDCs) under the action of TNF-α. As shown in FIG. 1, after MDDCs become mature via inducing, CD83, CD86 and CD11c were significantly increased, and the surface marker CD14 of monocytes and macrophages was significantly reduced.

### Example 2: Induced Isolation and Functional Identification of Killer CD8⁺ T Cells

After the antigen-loaded MDDCs and CD8⁺ T cells in peripheral blood were co-cultured, the antigen was presented to CD8⁺ T cells, so that the CD8⁺ T cells were activated to become antigen-specific killer CD8⁺ T cells, and then the pure killer CD8⁺ T cells were isolated through magnetic bead sorting and used for subsequent functional research. As can be seen from FIG. 2, compared with the non-activated CD8⁺ T cells, the killer CD8⁺ T cells had increased proliferation ability and enhanced cytokine-producing and secreting ability, and had specific killing ability against target cells.

### Example 3-1: Identification of TRABD2A Expression on the Surface of CD8⁺ T Cells

As can be seen in FIG. 3A, the killer CD8⁺ T cells exhibited TRABD2A metalloprotease expression as shown by RNA high throughput sequencing. The qRT-PCR quantification verified the expression of TRABD2A metalloprotease in CD8⁺ T cells. Detection results of cellular immunofluorescence and flow cytometry proved that TRABD2A was expressed on the cell membrane of CD8⁺ T cells.

### Example 3-2: Identification of TRABD2B Expression on the Surface of CD8⁺ T Cells

As can be seen from FIG. 3B, the qRT-PCR quantification verified the expression of TRABD2B metalloprotease in CD8⁺ T cells. Detection results of cellular immunofluorescence and flow cytometry proved that TRABD2B was expressed on the cell membrane of CD8⁺ T cells.

### Example 3-3: Identification of TRABD2A and TRABD2B Expression on the Surface of CD8⁺ T Cells

As can be seen from FIG. 3C, the qRT-PCR quantification verified the expression of TRABD2A and TRABD2B metalloproteases in CD8⁺ T cells. Detection results of cellular immunofluorescence and flow cytometry proved that TRABD2A was expressed on the cell membrane of CD8⁺ T cells.

### Example 4-1: Identification of Effect of Metal Cations on TRABD2A Protease Activity

Since TRABD2A is a metalloprotease and its activity is affected by metal ions, and the metal ion Mn²⁺ having an accelerating effect on TRABD2A metalloprotease activity and metal ions Co²⁺ and Ni²⁺ having an inhibiting effect were examined. The effect of the TRABD2A protein on CD8⁺ T cells was observed by the action of metal ions Mn²⁺, Co²⁺ and Ni²⁺ on TRABD2A metalloprotease on CD8⁺ T cells.

As shown in FIG. 4A, under the inhibiting effect of metal cations Co²⁺ and Ni²⁺ on the activity of TRABD2A protein, the tumor killing ability of CD8⁺ T cells was enhanced, and the killing ability was gradually enhanced along with the increase of the concentration. However, the tumor killing ability of CD8⁺ T cells was not enhanced under intervention with activity promoting cofactor Mn²⁺ of the TRABD2A protein. At a concentration of 20 μM, Co²⁺, Ni²⁺ and Mn²⁺ had little toxic effect on CD8⁺ T cells and target cells.

### Example 4-2: Identification of Effect of Metal Cations on TRABD2B Protease Activity

Since TRABD2B is a metalloprotease whose activity is affected by metal ions, metal ion Mn²⁺ that promotes the TRABD2B metalloprotease activity was examined. The effect of the TRABD2B protein on CD8⁺ T cells was observed by the action of metal ion Ni²⁺ upon TRABD2B metalloprotease on CD8⁺ T cells.

As shown in FIG. 4B, under the inhibiting effect of metal cation Ni²⁺ on the activity of TRABD2B protein, the tumor killing ability of CD8⁺ T cells was enhanced, and the killing ability was gradually enhanced along with the increase of the concentration. At concentration 20 μM, Ni²⁺ had little toxic effect on cells.

### Example 5-1: Design and Production of TRABD2A Monoclonal Antibody

In order to specifically block TRABD2A protein, a TRABD2A monoclonal antibody was designed and synthesized. The specific sequences of the antibody are set forth in SEQ ID NOs: 1-20.

The production steps of the antibody are as follows:
1. Cell line used: 293FT;
2. Culture medium used: 293FT medium;
3. Transfection reagent used: polyethylenimine (PEI);
4. Scheme for culturing 1 L cells:
(1) 293FT cells were cultured to reach a concentration of 2×10⁶ cells/mL 24 h prior to transfection;
(2) 1 mL of culture medium were transfected with 1 μg of antibody plasmid, and 1 L of culture cells were transfected with 1 mg of plasmid;
(3) 1 mg of plasmid DNA (0.67 mg of heavy chain plasmid and 0.33 mg of light chain plasmid) was mixed, and 50 mL of Opti-MEM medium was added;
(4) 6 mg of PEI transfection reagent was mixed with 50 mL of Opti-MEM medium for 3 min at room temperature;
(5) the plasmid solution of the step (3) and the PEI solution of the step (4) were well mixed and left to stand at room temperature for 15 min;
(6) the mixed solution of the step (5) was added into a cell culture medium;
(7) the mixed solution of cells was incubated in an incubator at 37° C. and shaken at 125 rpm;
(8) After 144 h of transfection, cell culture fluid was harvested and cells were removed.

The purification steps of the antibody are as follows:
1. Binding buffer used: lx PBS, pH 7.4 (KH₂PO₄ 2 mM; Na₂HPO₄ 8 mM; NaCl 136 mM; KCl 2.6 mM);
2. Elution buffer used: 50 mM acetic acid, pH 3.0;
3. Purification steps used:
(1) Protein A column was equilibrated with binding buffer;
(2) The cell supernatant was centrifuged at 2000 rpm for 5 min to remove the cells, and the resulting supernatant was centrifuged at 13000 rpm for 20 min, wherein the centrifugation temperature was required to be 4° C. for both cases;
(3) the resulting supernatant was loaded onto the protein A column;
(4) the antibody-bound column was washed three times with binding buffer;
(5) the column was washed with 50 mM eluent to obtain an antibody solution;
(6) the resulting antibody solution was centrifuged at 13000 rmp for 10 min, wherein the temperature was required to be 4° C.;
(7) the protein concentration was measured using A₂₈₀ OD.

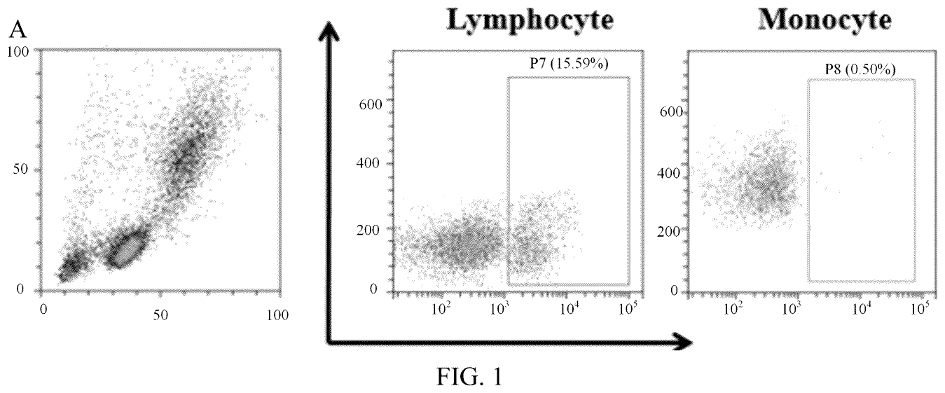

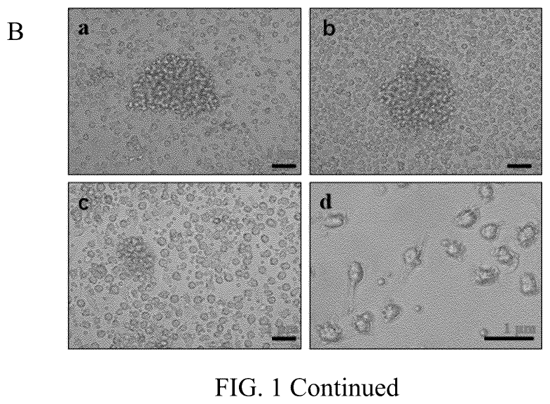

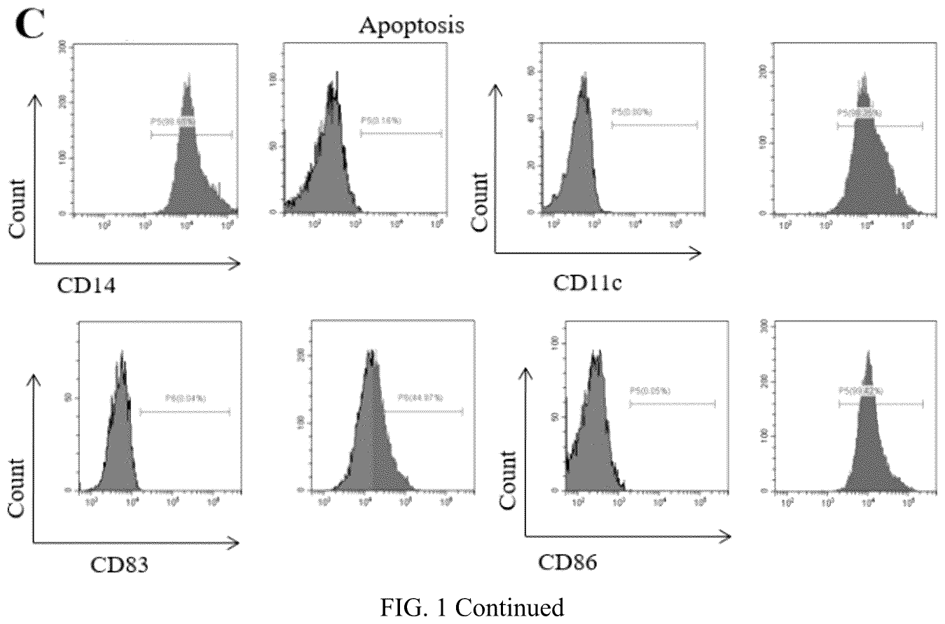

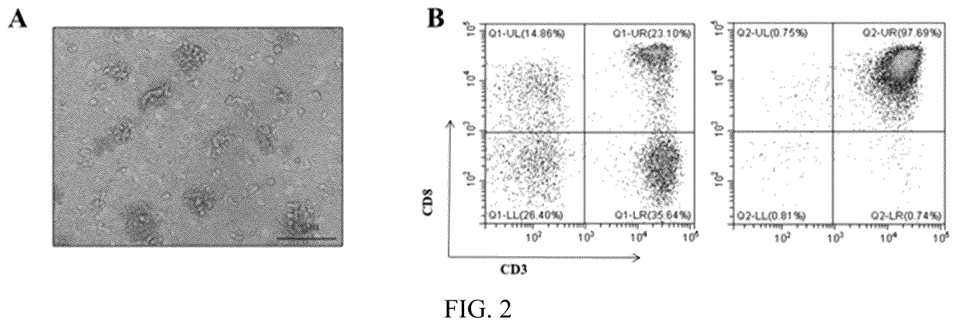

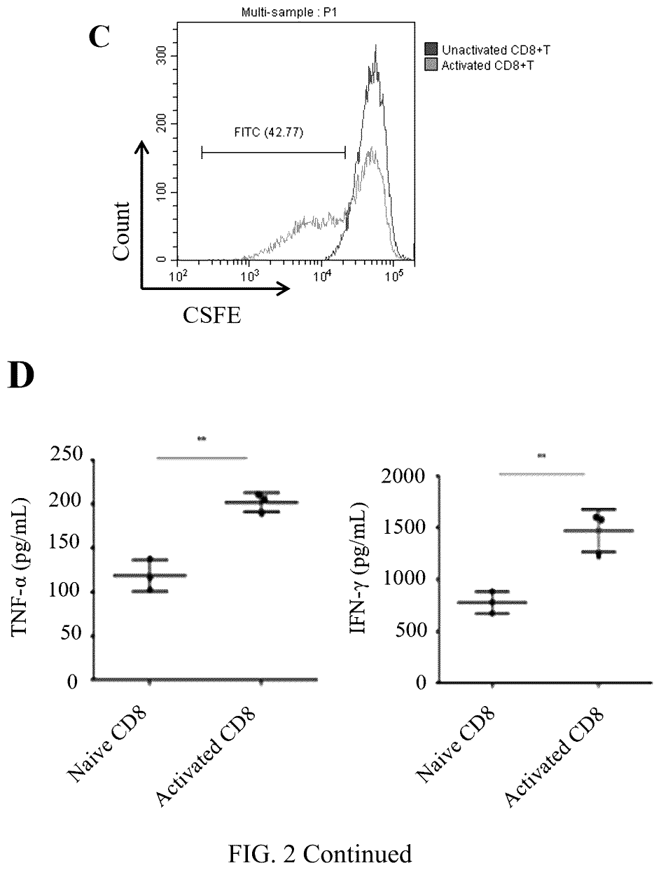

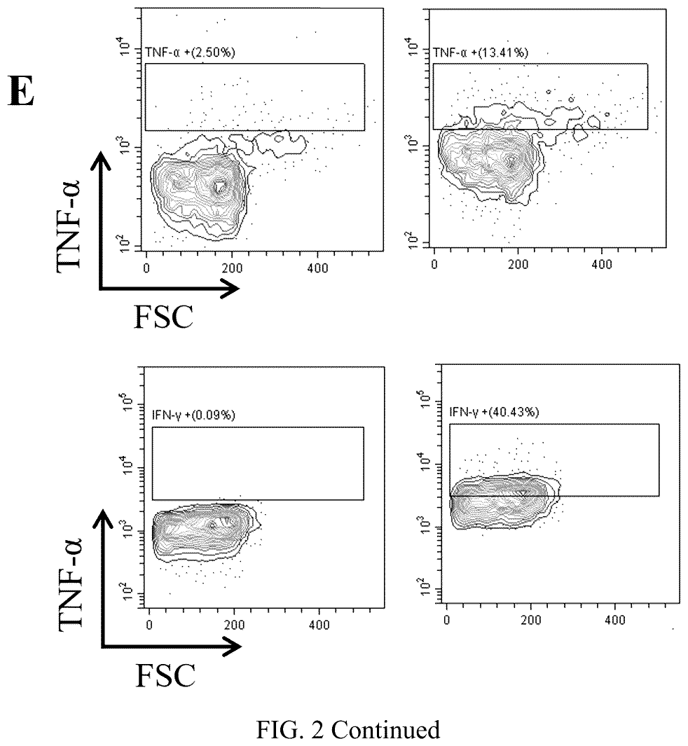

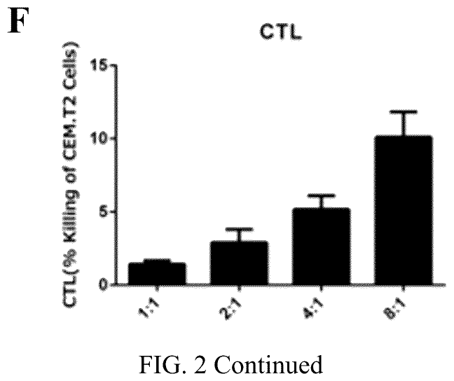

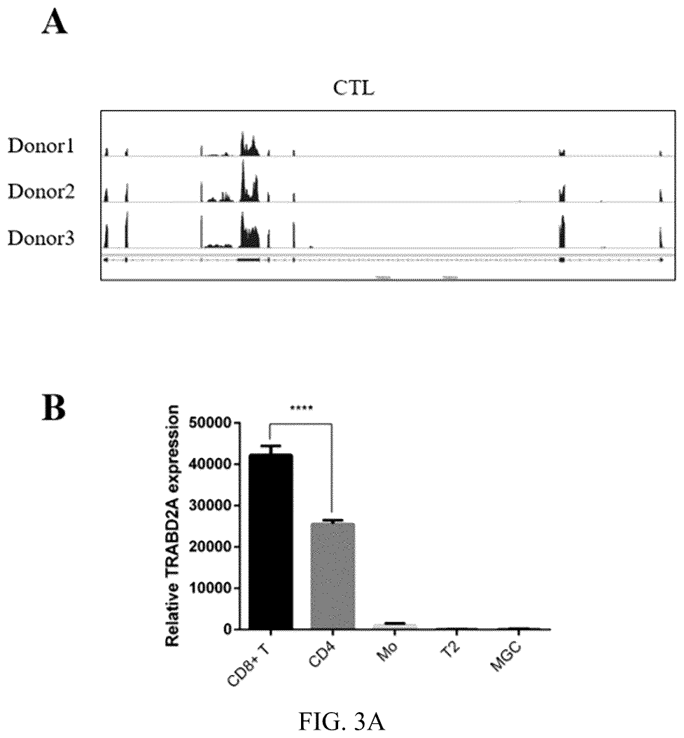

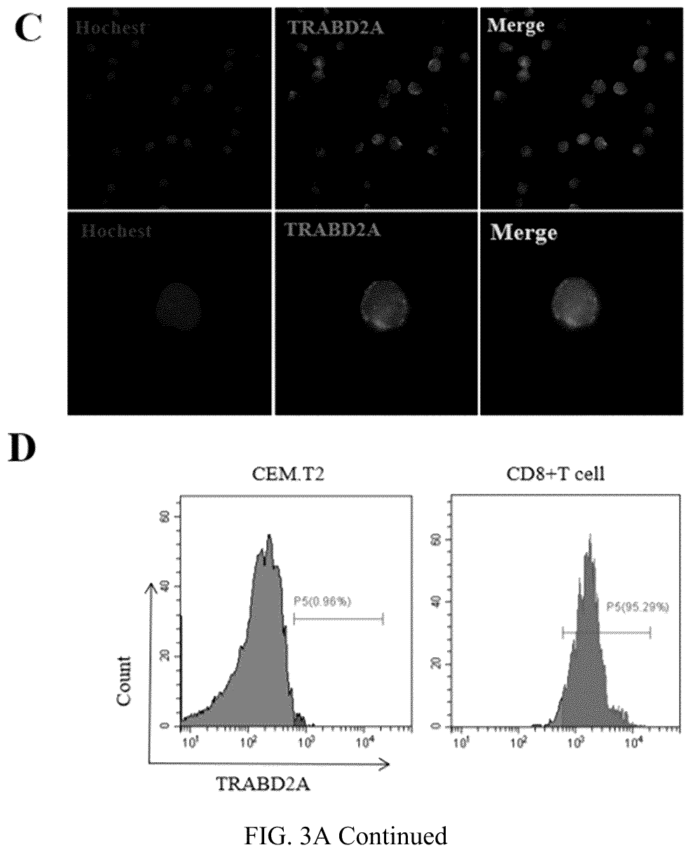

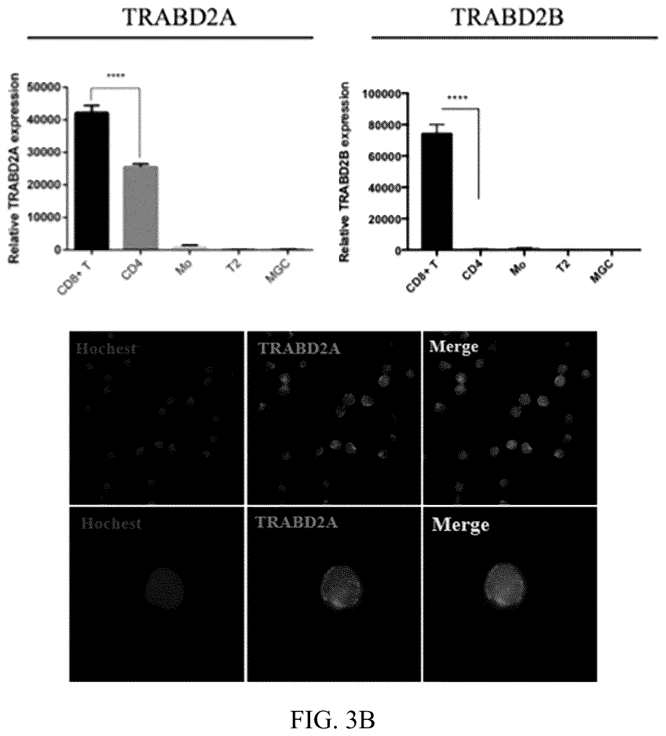

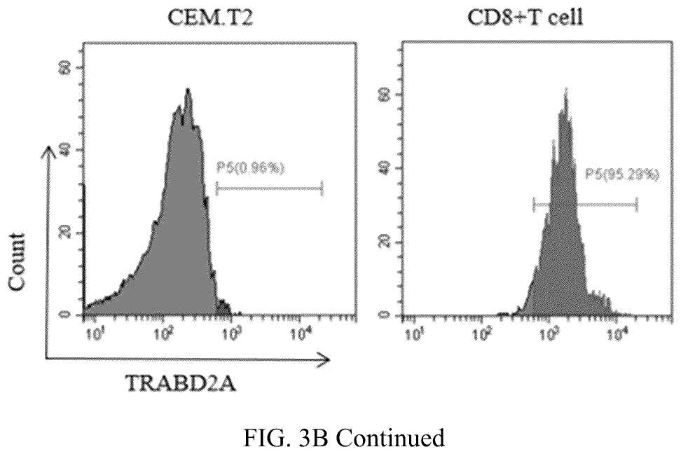

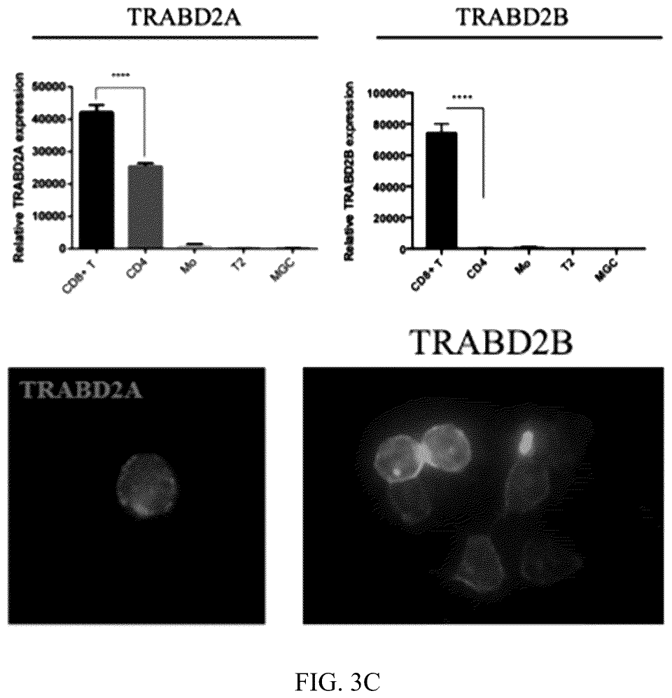

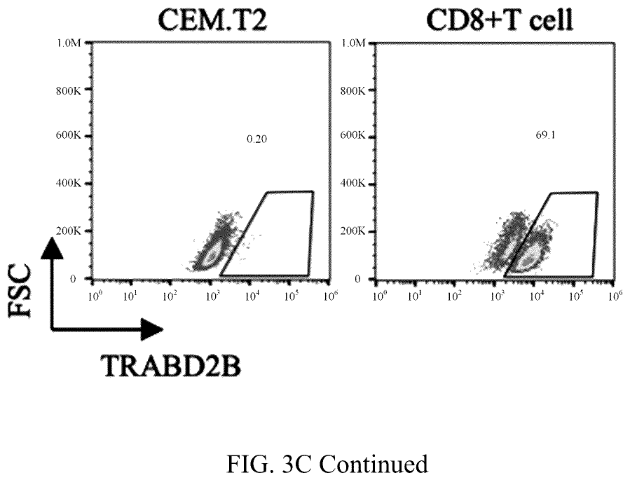

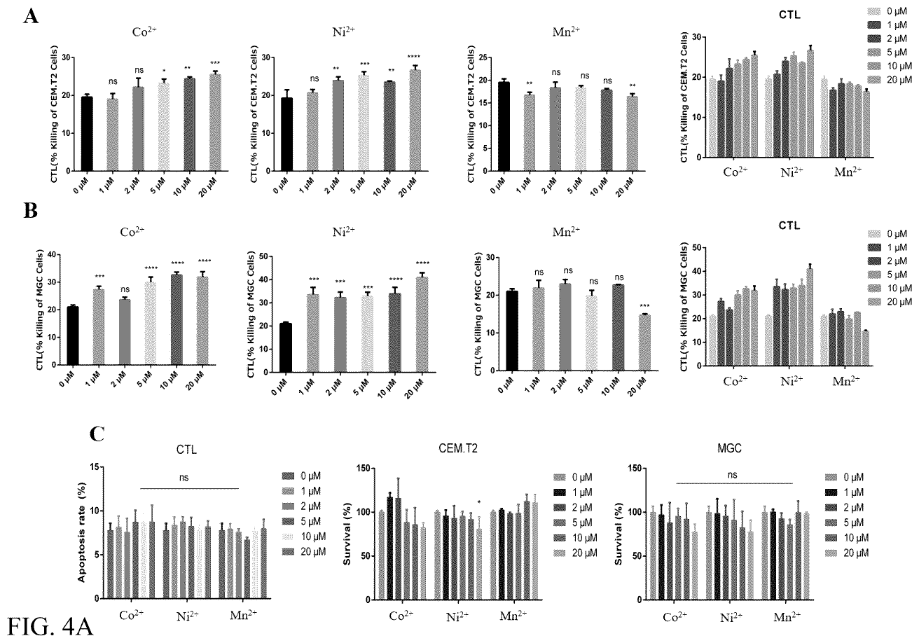

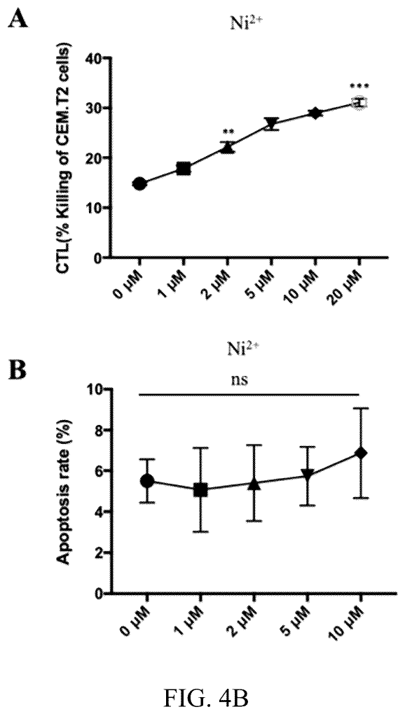

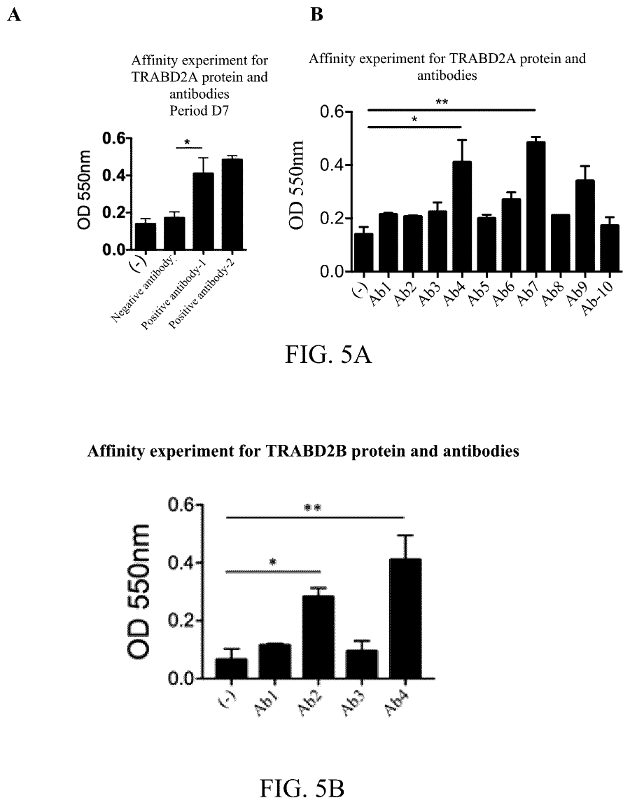

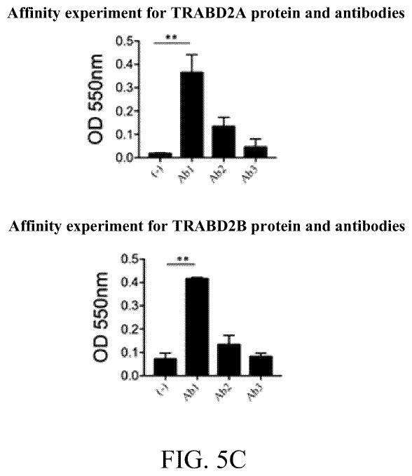

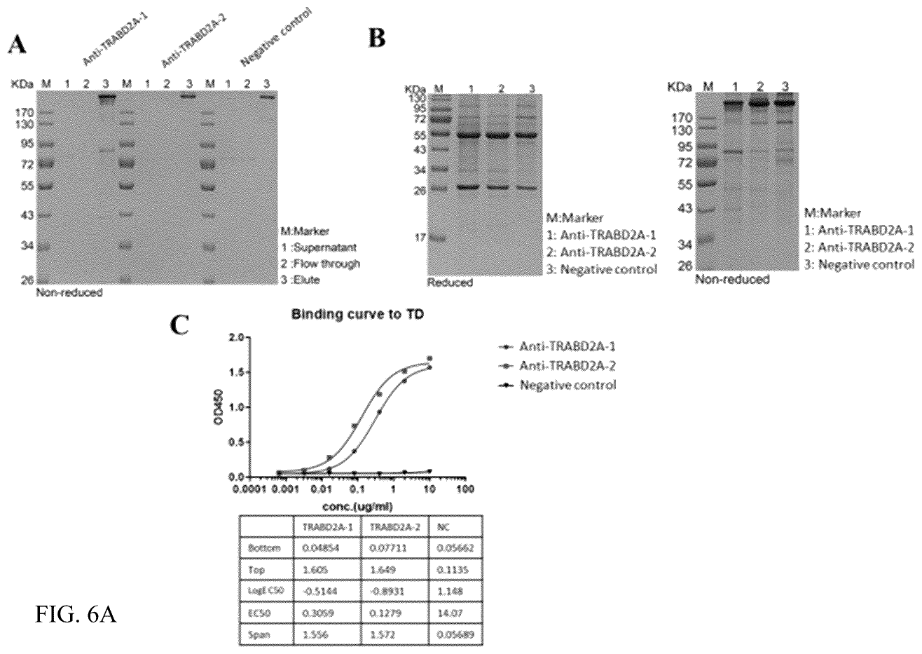

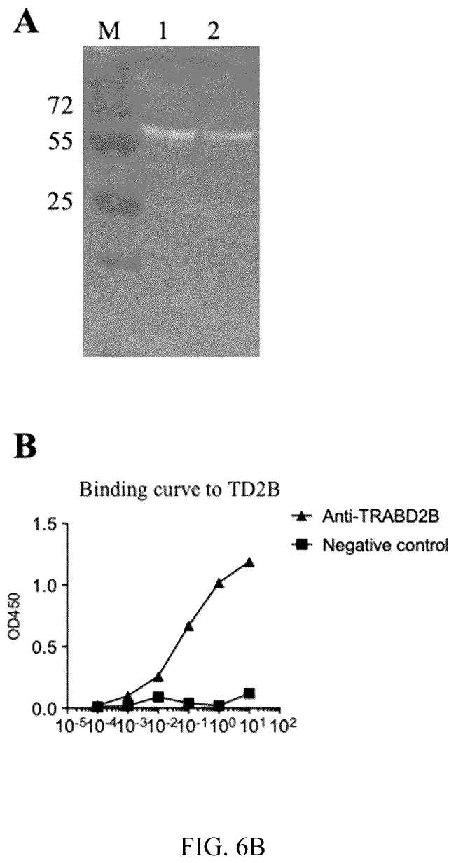

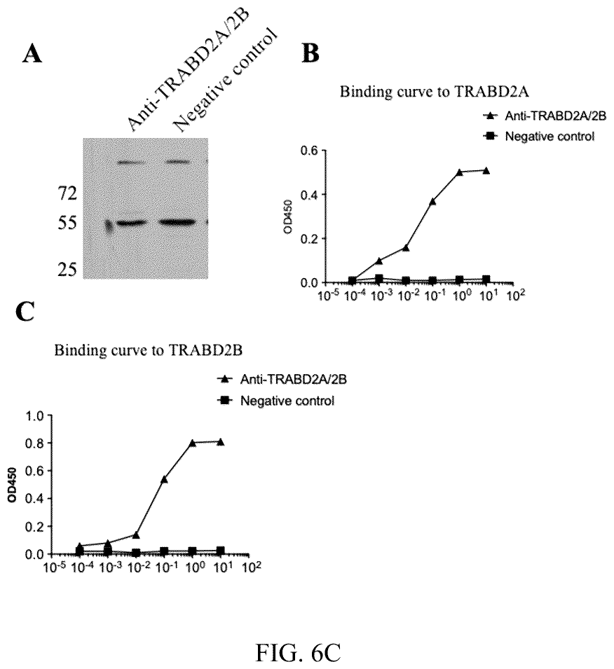

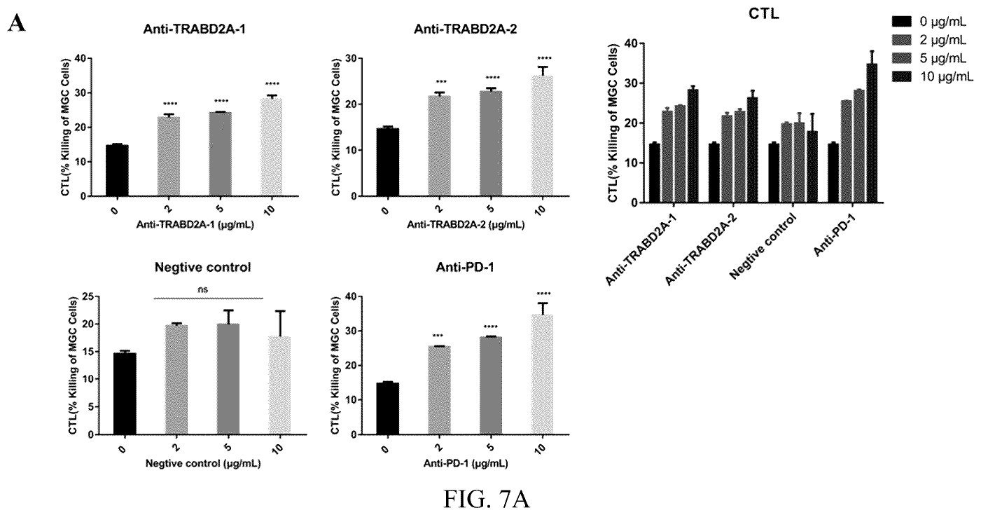

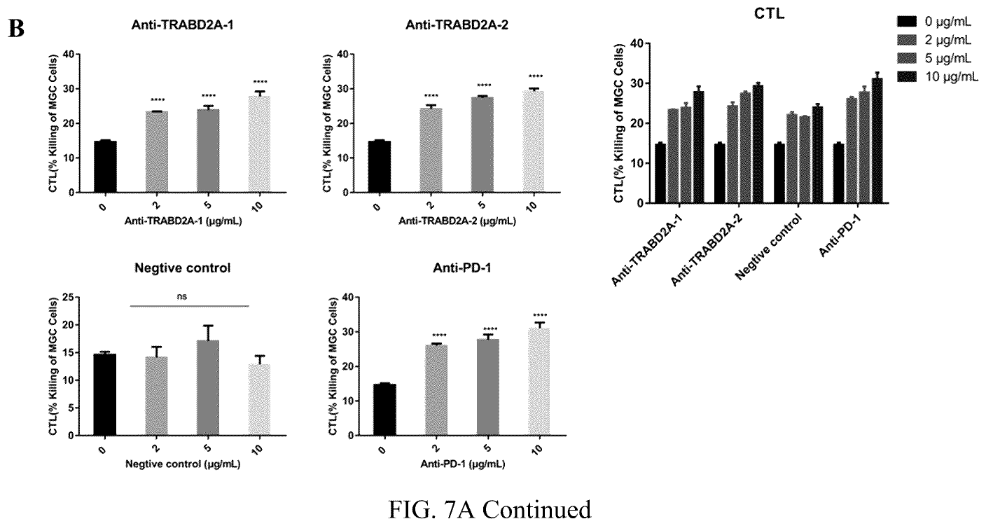

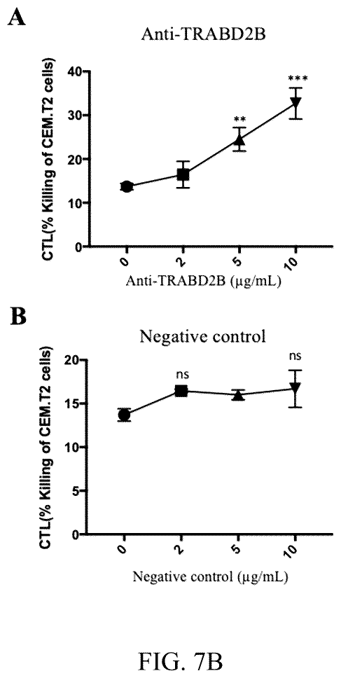

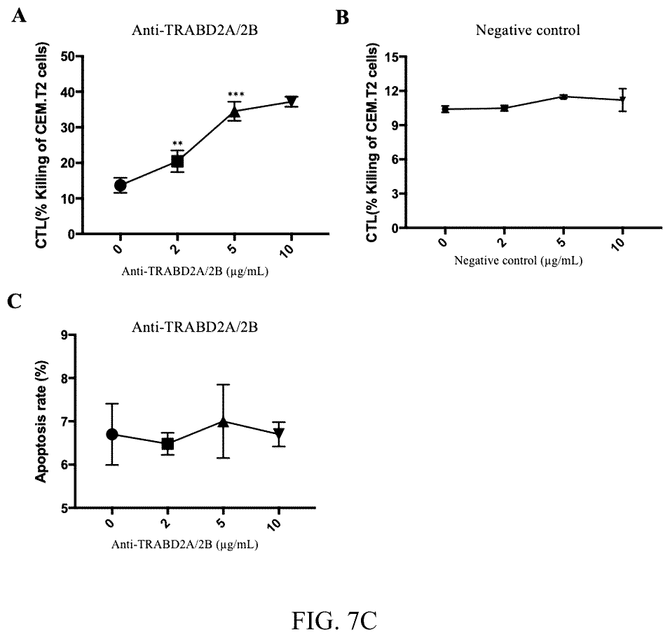

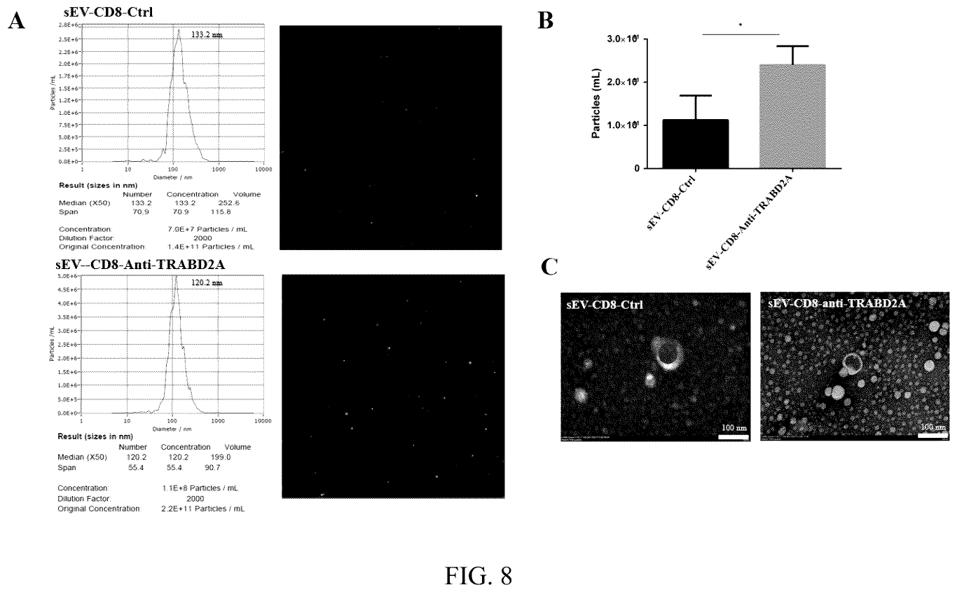

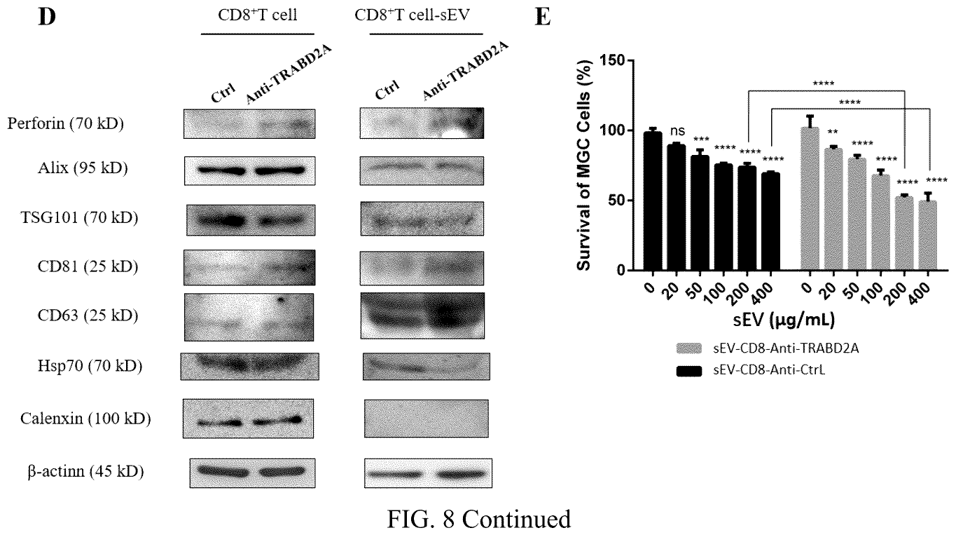

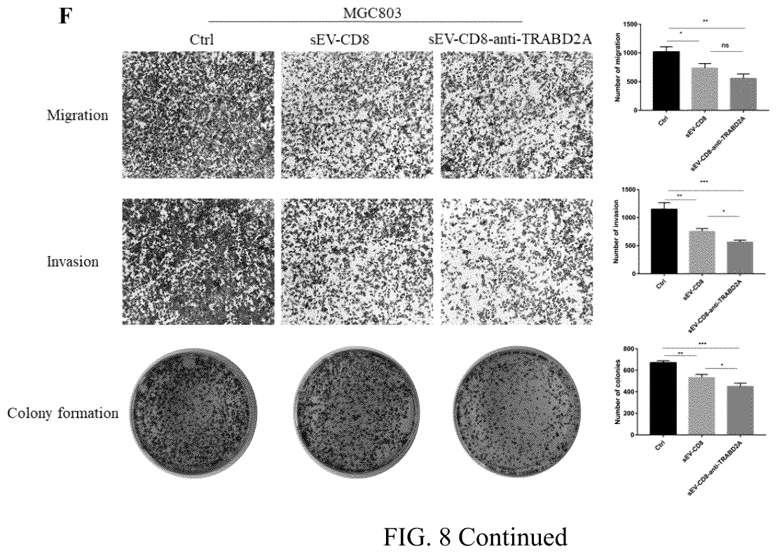

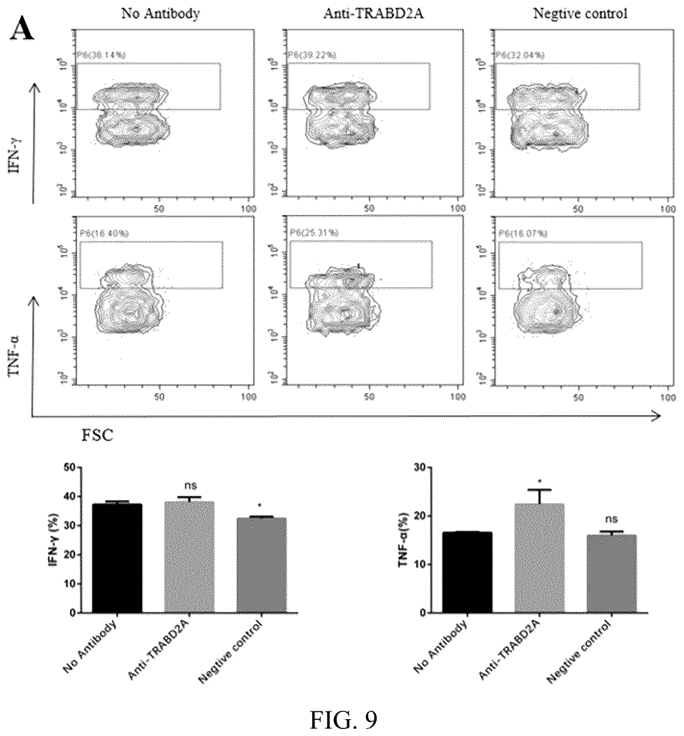

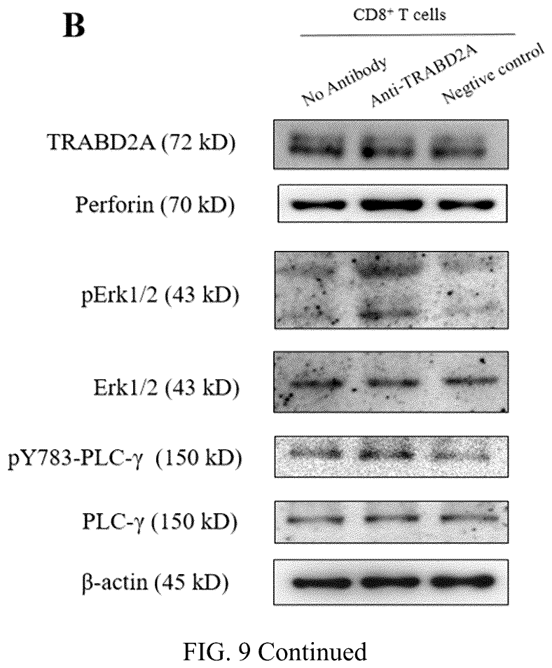

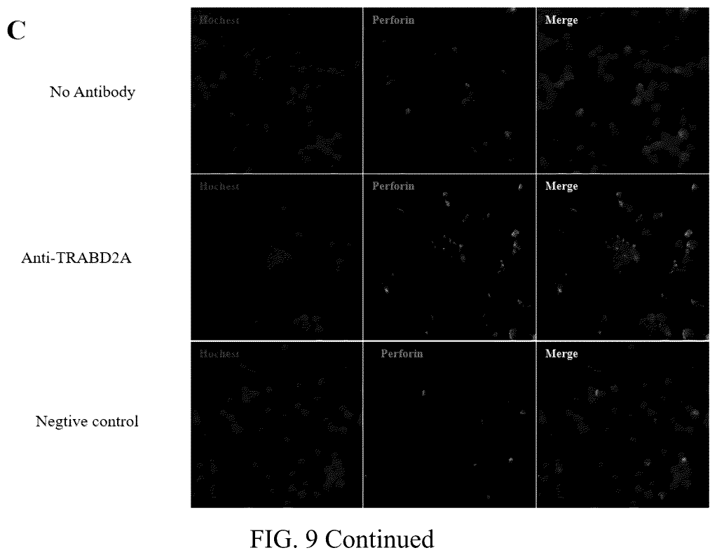

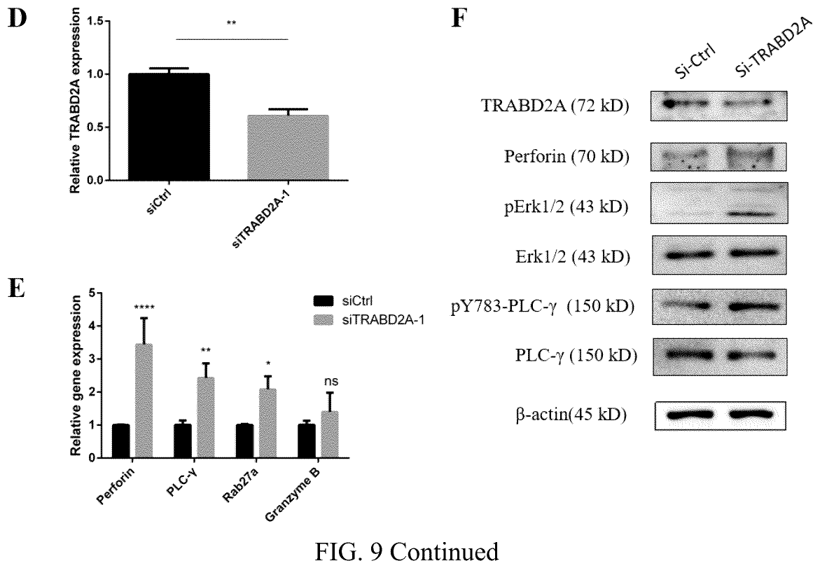

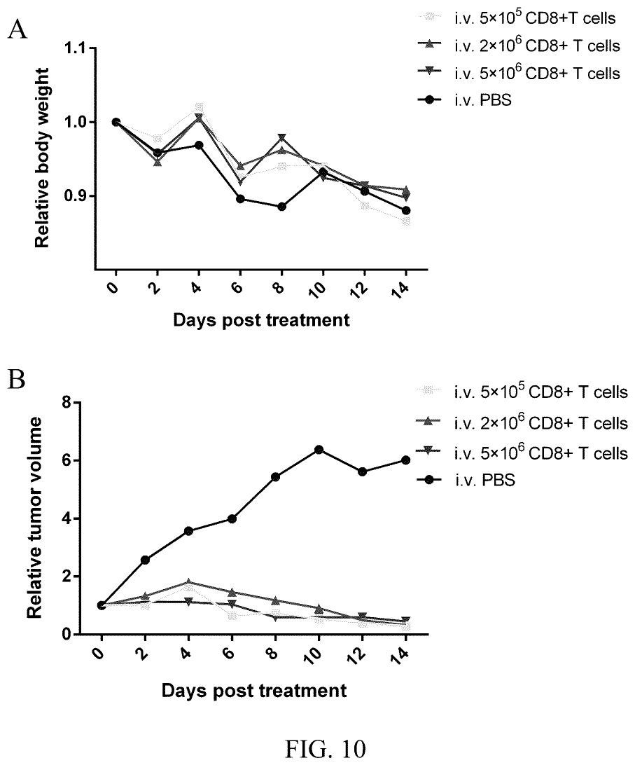

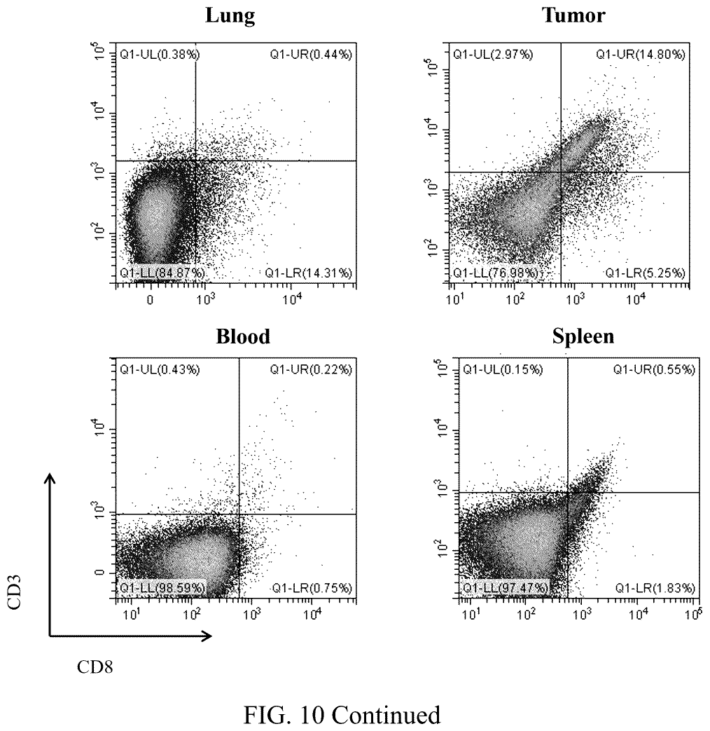

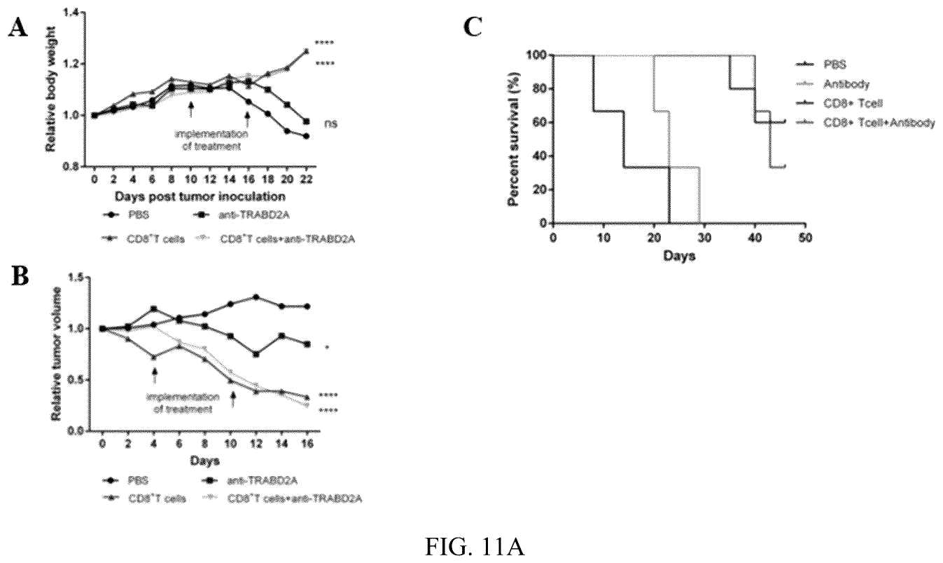

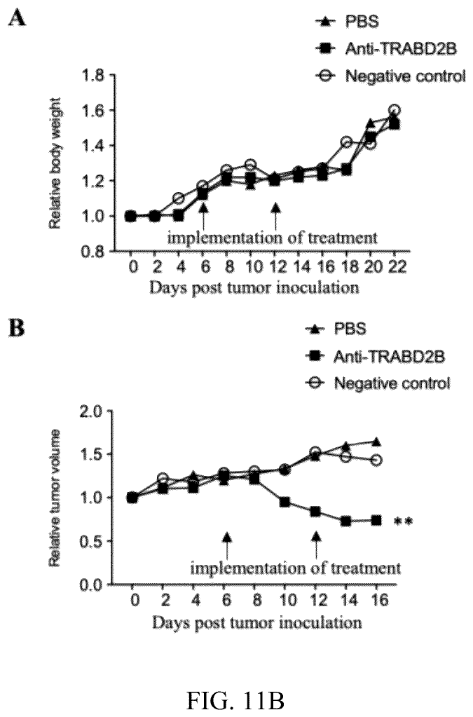

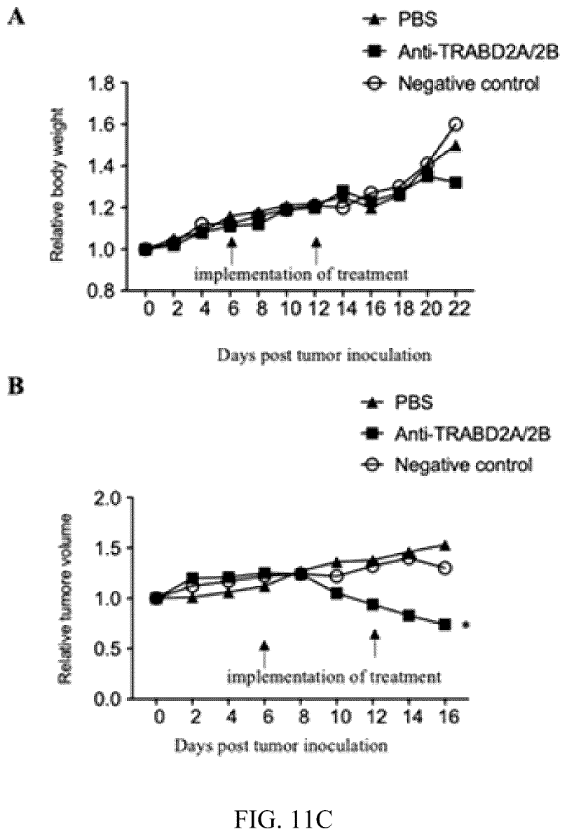

The affinity experiment for the TRABD2A protein and antibodies is shown in FIG. 5A.

### Example 5-2: Design and Production of TRABD2B Monoclonal Antibody

In order to specifically block TRABD2B protein, a TRABD2B monoclonal antibody was designed and synthesized. The specific sequences of the antibody are set forth in SEQ ID NOs: 21-28.

The production steps of the antibody are as follows:
1. Cell line used: 293FT;
2. Culture medium used: 293FT medium;
3. Transfection reagent used: polyethylenimine (PEI);
4. Scheme for culturing 1 L cells:
(1) 293FT cells were cultured to reach a concentration of 2×10⁶ cells/mL 24 h prior to transfection;
(2) 1 mL of culture medium were transfected with 1 μg of antibody plasmid, and 1 L of culture cells were transfected with 1 mg of plasmid;
(3) 1 mg of plasmid DNA (0.67 mg of heavy chain plasmid and 0.33 mg of light chain plasmid) was mixed, and 50 mL of Opti-MEM medium was added;
(4) 6 mg of PEI transfection reagent was mixed with 50 mL of Opti-MEM medium for 3 min at room temperature;
(5) the plasmid solution of the step (3) and the PEI solution of the step (4) were well mixed and left to stand at room temperature for 15 min;
(6) the mixed solution of the step (5) was added into a cell culture medium;
(7) the mixed solution of cells was incubated in an incubator at 37° C. and shaken at 125 rpm;
(8) After 144 h of transfection, cell culture fluid was harvested and cells were removed.

The purification steps of the antibody are as follows:
1. Binding buffer used: lx PBS, pH 7.4 (KH₂PO₄ 2 mM; Na₂HPO₄ 8 mM; NaCl 136 mM; KCl 2.6 mM);
2. Elution buffer used: 50 mM acetic acid, pH 3.0;
3. Purification steps used:
(1) Protein A column was equilibrated with binding buffer;
(2) The cell supernatant was centrifuged at 2000 rpm for 5 min to remove the cells, and the resulting supernatant was centrifuged at 13000 rpm for 20 min, wherein the centrifugation temperature was required to be 4° C. for both cases;
(3) the resulting supernatant was loaded onto the protein A column;
(4) the antibody-bound column was washed three times with binding buffer;
(5) the column was washed with 50 mM eluent to obtain an antibody solution;
(6) the resulting antibody solution was centrifuged at 13000 rmp for 10 min, wherein the temperature was required to be 4° C.;
(7) the protein concentration was measured using A₂₈₀ OD.

The affinity experiment for the TRABD2B protein and antibodies is shown in FIG. 5B.

### Example 5-3: Preparation of Bispecific Antibody for Inhibiting Both TRABD2A and TRABD2B Protease Activity

In order to specifically block both TRABD2A and TRABD2B proteins, a bispecific antibody capable of recognizing both TRABD2A and TRABD2B, and a negative control antibody are designed and synthesized. The specific sequences of the antibody are set forth in SEQ ID NOs: 29-34.

The production steps of the antibody are as follows:
1. Cell line used: 293FT;
2. Culture medium used: 293FT medium;
3. Transfection reagent used: polyethylenimine (PEI);
4. Scheme for culturing 1 L cells:
(1) 293FT cells were cultured to reach a concentration of 2×10⁶ cells/mL 24 h prior to transfection;
(2) 1 mL of culture medium were transfected with 1 μg of antibody plasmid, and 1 L of culture cells were transfected with 1 mg of plasmid;
(3) 1 mg of plasmid DNA (0.67 mg of heavy chain plasmid and 0.33 mg of light chain plasmid) was mixed, and 50 mL of Opti-MEM medium was added;
(4) 6 mg of PEI transfection reagent was mixed with 50 mL of Opti-MEM medium for 3 min at room temperature;
(5) the plasmid solution of the step (3) and the PEI solution of the step (4) were well mixed and left to stand at room temperature for 15 min;
(6) the mixed solution of the step (5) was added into a cell culture medium;
(7) the mixed solution of cells was incubated in an incubator at 37° C. and shaken at 125 rpm;
(8) After 144 h of transfection, cell culture fluid was harvested and cells were removed.

The purification steps of the antibody are as follows:
1. Binding buffer used: lx PBS, pH 7.4 (KH₂PO₄ 2 mM; Na₂HPO₄ 8 mM; NaCl 136 mM; KCl 2.6 mM);
2. Elution buffer used: 50 mM acetic acid, pH 3.0;
3. Purification steps used:
(1) Protein A column was equilibrated with binding buffer;
(2) The cell supernatant was centrifuged at 2000 rpm for 5 min to remove the cells, and the resulting supernatant was centrifuged at 13000 rpm for 20 min, wherein the centrifugation temperature was required to be 4° C. for both cases;
(3) the resulting supernatant was loaded onto the protein A column;
(4) the antibody-bound column was washed three times with binding buffer;
(5) the column was washed with 50 mM eluent to obtain an antibody solution;
(6) the resulting antibody solution was centrifuged at 13000 rmp for 10 min, wherein the temperature was required to be 4° C.;
(7) the protein concentration was measured using A₂₈₀ OD.

The affinity experiment for the TRABD2A and TRABD2B proteins and bispecific antibodies is shown in FIG. 5C.

### Example 6-1: Capability Verification of Improving Target Cell Killing Ability of CD8⁺ T Cells by Inhibiting TRABD2A Protein

Three antibodies anti-TRABD2A-1 (amino acid sequences of heavy and light chains are set forth in SEQ ID NOs: 7 and 8, respectively), anti-TRABD2A-2 (amino acid sequences of heavy and light chains are set forth in SEQ ID NOs: 13 and 14, respectively) and negative control (amino acid sequences of heavy and light chains are set forth in SEQ ID NOs: 19 and 20, respectively) synthesized in Example 5-1 were used. The synthesized antibodies were subjected to affinity purification by using Mabselect Sure, and the analysis result of non-reduced SDS-PAGE showed that in the final purified liquid, the protein structure was complete and the purification effect was good. The final SDS-PAGE results of the three antibodies showed that a remarkable band was formed at 55 kD of the anti-TRABD2A synthetic antibody, and the protein had antigen cross reaction at 26 kD. After detection with antigen, it was shown that anti-TRABD2A-1 and anti-TRABD2A-2 exhibited high binding efficiency, while the negative control antibody did not bind to the antigen. After concentration of the antibodies, the protein concentrations were measure by A₂₈₀, and the concentrations of anti-TRABD2A-1, anti-TRABD2A-2 and negative control were 0.92 mg/mL, 1.6 mg/mL and 2.98 mg/mL, respectively. The proteins were snap frozen in liquid nitrogen and stored at −80° C. for later use. The quality detection of the TRABD2A antibodies after synthesis is shown in FIG. 6A.

The effect of Anti-TRABD2A-1 and Anti-TRABD2A-2 antibodies with the TRABD2A protein specifically blocked and that of the negative control antibody on target cell killing ability of CD8⁺ T cells were examined. As shown in FIG. 7A, anti-TRABD2A-1 and anti-TRABD2A-2 with the TRABD2A blocked had enhanced effect on target cell killing ability of CD8⁺ T cells, while the negative control antibody had little effect on target cell killing function of CD8⁺ T cells. Two modes were adopted for intervening with TRABD2A protein on CD8⁺ T cells, and the difference of the two modes was that the CD8⁺ T cells, after incubation with the antibody, kill the target cells with residual blocking antibody removed or unremoved. The killing results showed that the two modes had similar effect on the target cell killing ability of CD8⁺ T cells, which shows that the three TRABD2A antibodies have strong antigen-binding ability and good blocking effect. Free antibodies had no effect on target cells, demonstrating that TRABD2A antibody had little toxic effect on target cells at different concentrations.

### Example 6-2: Capability Verification of Improving Target Cell Killing Ability of CD8⁺ T Cells by Inhibiting TRABD2B Protein

The anti-TRABD2B-1 (amino acid sequences of heavy and light chains are set forth in SEQ IN NOs: 23 and 24, respectively), the anti-TRABD2B-2 (amino acid sequences of heavy and light chains are set forth in SEQ IN NOs: 27 and 28, respectively) and negative control (amino acid sequences of heavy and light chains are set forth in SEQ IN NOs: 21 and 22, respectively) antibodies synthesized in Example 5-2 were used. The synthesized antibodies were subjected to affinity purification by using Mabselect Sure, and the analysis result of non-reduced SDS-PAGE showed that in the final purified liquid, the protein structure was complete and the purification effect was good. The final SDS-PAGE results of the two antibodies showed that a remarkable band was formed at 55 kD of the anti-TRABD2B synthetic antibody. After detection with antigen, it was shown that anti-TRABD2B exhibited high binding efficiency, while the negative control antibody did not bind to the antigen. After the antibodies were concentrated, the protein concentration of anti-TRABD2B was 1.22 mg/mL and that of the negative control was 1.29 mg/mL, as measured by A₂₈₀. The proteins were snap frozen in liquid nitrogen and stored at −80° C. for later use. The quality detection of the TRABD2B antibody after synthesis is shown in FIG. 6B.

The effect of the anti-TRABD2B antibody that specifically blocks TRABD2B protein and that of the negative control antibody on the killing of target cells by CD8⁺ T cells were examined. As shown in FIG. 7B, anti-TRABD2B antibody that blocks TRABD2B had an enhancing effect on the killing of target cells by CD8⁺ T cells, while the negative control antibody had little effect on the target cell killing function of CD8⁺ T cells.

### Example 6-3: Capability Verification of Significantly Improving Target Cell Killing Ability of CD8⁺ T Cells by Inhibiting Both TRABD2A and TRABD2B Proteins

The bispecific antibody (amino acid sequences of heavy and light chains are set forth in SEQ IN NOs: 29 and 30, respectively) and negative control (amino acid sequences of heavy and light chains are set forth in SEQ IN NOs: 33 and 24, respectively) antibody synthesized in Example 5-3 were used. The synthesized antibodies were subjected to affinity purification by using Mabselect Sure, and the analysis result of non-reduced SDS-PAGE showed that in the final purified liquid, the protein structure was complete and the purification effect was good. The final SDS-PAGE results of the antibodies showed that a remarkable band was formed at 55 kD of the anti-TRABD2A/2B bispecific synthetic antibody. After detection with antigen, it was shown that the anti-TRABD2A/2B bispecific antibody was capable of binding to both TRABD2A and TRABD2B, while the negative control antibody did not bind to both the antigens. After the antibodies were concentrated, the protein concentration of anti-TRABD2A/2B was 1.23 mg/mL and that of the negative control was 1.02 mg/mL, as measured by A₂₈₀. The proteins were snap frozen in liquid nitrogen and stored at −80° C. for later use. The quality detection of the anti-TRABD2A/2B bispecific antibody after synthesis is shown in FIG. 6C.

The effect of the bispecific antibody blocking both TRABD2A and TRABD2B proteins on target cell killing ability of CD8⁺ T cells were examined. As shown in FIG. 7C, the bispecific antibody that blocks both TRABD2A and TRABD2B had an enhancing effect on the killing of target cells by CD8⁺ T cells, while the negative control had little effect on the target cell killing function of CD8⁺ T cells.

### Example 7: Increase in Secretion of CD8⁺ T Cell-Derived Small Extracellular Vesicles after TRABD2A Antibody Blockade

In order to verify whether TRABD2A has the same effect on the generation and release of small extracellular vesicles in cells as its inhibition effect on the generation and release of viral particles in viruses, cell culture supernatants of CD8⁺ T cells were collected after blocking with TRABD2A antibody and negative control antibody. The same volume of supernatant was taken, and after extracting small extracellular vesicles using a kit, quality evaluation was performed on the small extracellular vesicles. As shown in FIG. 8, after being blocked with TRABD2A antibody, the CD8⁺ T cells produced small extracellular vesicles with a size of about 120.2 nm and the concentration was about 2.2×10¹¹ particles/mL, whereas after being blocked with negative control antibody, the CD8⁺ T cells produced small extracellular vesicles with a size of about 133.2 nm, and the concentration was about 1.4×10¹¹ particles/mL. In comparison, the release of CD8⁺ T cell-derived small extracellular vesicles was promoted after the TRABD2A protein on the surface of CD8⁺ T cells was blocked with the TRABD2A antibody. Western blotting analysis was performed on these two kinds of small extracellular vesicles, and surprisingly, at both cellular and small extracellular vesicle levels, the expression amount of perforin protein in CD8⁺ T cells blocked with the TRABD2A antibody are higher than that of the control group. Results of killing target cells by using these two kinds of small extracellular vesicles showed that both of them had concentration-dependent toxicity on the target cells, and more particularly, at the concentrations of 200 μg/mL and 400 μg/mL, the small extracellular vesicles generated by blocking CD8⁺ T cells with TRABD2A antibody had higher toxicity on the target cells than those of the control group. This indicates that after the CD8⁺ T cells are blocked with TRABD2A antibody, the toxic effect is higher than the control group at high concentrations. Transwell migration and invasion experiments showed that compared with sEVs in the control group, sEVs collected before and after TRABD2A antibody blocking all reduced the migration and invasion level of tumor cells, wherein the invasion effect of the tumor cells treated with sEVs collected after CD8⁺ T cells were blocked with TRABD2A antibody was significantly lower than that of sEVs collected without antibody blocking, and it was proved that sEVs generated by CD8⁺ T after TRABD2A blocking had a better invasion inhibition effect on the tumor cells. The plate colony formation experiment also proved that sEVs generated by CD8⁺ T cells could inhibit the colony formation of tumor cells, and sEVs generated by CD8⁺ T cells after TRABD2A blocking had better inhibition effect on tumors.

### Example 8: Effect of Blocking TRABD2A Protein on Functions of CD8⁺ T Cells

To explore the reason why blocking the TRABD2A protein improves the target cell killing ability of CD8⁺ T cells, the functional status of CD8⁺ T cells after blocking with the TRABD2A antibody and negative control antibody was examined. The flow cytometer was used for detecting the intracellular IFN-7 and TNF-α expression of CD8⁺ T cells. As shown in FIG. 9, after the cells were blocked with TRABD2A antibody, the intracellular TNF-α level was significantly increased. The intracellular TNF-α includes transmembrane TNF-α (mTNF-α) and secretory TNF-α (sTNF-α), and the generation of the sTNF-α requires that the mTNF-α is cut under the action of membrane metalloprotease. However, whether TRABD2A participates in this process and whether the activity of TRABD2A in cutting the mTNF-α is improved after binding of TRABD2A antibody have not been testified. However, as can be seen from the Western immunoblotting results, the CD8⁺ T cells blocked with TRABD2A antibody had higher perforin level and increased phosphorylation level of the PLC pathway associated with perforin polarization and the Erk pathway for secretion of associated vesicles, as compared to the control group. Immunofluorescence results also showed that CD8⁺ T cells blocked with TRABD2A antibody had significantly increased perforin expression and polarization, as compared to the control group. To further verify, TRABD2A gene was knocked out by siRNA, and qRT-PCR and Western immunoblotting results showed that after TRABD2A was knocked out, the gene expression levels of PLC-7 and Rab27a were increased, and Rab27a played an important role in mediating the secretion of vesicles at late stage to the membrane, and thus the expression level of perforin protein was increased. The above results all indicate that after blocking with TRABD2A antibody, the CD8⁺ T cells had increased vesicle secretion level and increased polarization release level of the perforin, and the perforin plays an important role in killing target cells by CD8⁺ T cells.

### Example 9-1: Animal Experiments

In order to examine the function of CD8⁺ T cells in killing tumor cells in vivo after intervention with TRABD2A antibody, nude mice implanted with solid tumor MGC-803 were selected as research objects for adoptive CD8⁺ T cell treatment experiments.

Firstly, MGC-803 was amplified in vitro and was resuspended in PBS at a concentration of 5×10⁴/μL, and 200 μL of tumor cell suspension was transplanted into the nude mice by means of subcutaneous injection, thus creating subcutaneous tumor. When the tumor volume reached 4-6 mm³ (day 0), treatment with CD8⁺ T cells cultured in vitro that had killing ability against MGC cells was performed. After modeling, 3 treatment experimental groups and 1 PBS control group were set up for dose exploring experiment in cell treatment. The treatment doses of the experimental groups were 5×10⁵, 2×10⁶ and 5×10⁶, respectively, and CD8⁺ T cells were injected through the tail vein on day 0 and day 6 for treatment. As shown in FIG. 10, the change in the body weight of the nude mice after the treatment relative to the day 0 was recorded in FIG. 10A, and the results showed that the body weight of mice in the PBS group without cell treatment showed a continuous decrease trend and a sharp decrease, and the cell treatment groups with different concentrations also showed a continuous decrease trend, and the decrease of the body weight in the groups 2×10⁶ and 5×10⁶ at the end of the treatment (day 14) was lower than that of the control group and the low dose group (5×10⁵). FIG. 10B records the change in tumor volume of nude mice relative to day 0. The results showed that the PBS control group without cell treatment showed significant increase in tumor volume over time, while the three cell treatment groups at different doses showed little change relative to day 0 and the tumor volume remained stable at a size slightly smaller than the initial tumor volume. Finally, the optimal 2×10⁶ CD8⁺ T cell treatment dose group was selected for subsequent animal treatment experimental study. FIG. 10C shows the results of CD8⁺ T cell flow cytometry analysis of blood, spleen, lung and tumor tissues obtained by dissection of nude mice in the 2×10⁶ CD8⁺ T cell treatment group after treatment. Different tissues were each digested into cell suspension under the action of tissue digestive enzyme, and after red blood cell lysis treatment, the cell suspension was filtered through a 100 μm cell sieve to form single cell suspension, which was then stained using CD8 and CD3 antibodies and finally loaded for flow cytometry analysis. The results showed that after two rounds of treatment with CD8⁺ T cells, no or only trace amount of CD8⁺ T cells remained in the blood and lungs of nude mice, and CD8⁺ T cells mostly entered the tumor tissue and a small fraction was detected in the spleen. This indicates that the used killer CD8⁺ T cells targeting MGC-803 can reach the tumor site through blood circulation after entering the nude mice via tail vein injection, thereby playing its role.

After exploring of the treatment dose of CD8⁺ T cells for the MGC-loaded tumor models, 2×10⁶ was selected as the final treatment dose, and the second in vivo animal study was performed, in which intervention with TRABD2A antibody was involved.

In the experiment, there were 3 experimental groups and 1 control group, namely TRABD2A antibody single treatment group, CD8⁺ T cell treatment group, treatment group of CD8⁺ T cells co-incubated with TRABD2A antibody, and PBS control group. As shown in FIG. 11A, FIG. 11AA showed the change in body weight of nude mice relative to tumor bearing day (day 0), and according to FIG. 11AA, the first and second treatments were performed on day 10 and day 16, respectively. The results showed that the body weight of nude mice treated with CD8⁺ T cells and CD8⁺ T cells co-incubated with TRABD2A antibody was increased constantly, while that of nude mice treated with TRABD2A antibody alone and those in PBS control group tended to decrease constantly after treatment. FIG. 11AB shows the change in tumor volume of nude mice relative to 4 days before treatment (day 0). According to FIG. 11AB, two treatments were performed on day 4 and day 10, respectively (equivalent to day 10 and day 16 in FIG. 11AA). The results showed that, after treatment, a significant decrease was observed in tumor volume of nude mice treated with CD8⁺ T cells and CD8⁺ T cells co-incubated with TRABD2A antibody, but no significant difference was present between the two treatment modalities; the TRABD2A antibody single treatment group showed a slight downward trend, while the PBS control group showed an increasing tumor volume of nude mice, which eventually maintained stable. FIG. 11AC shows analysis of survival curves of four groups of tumor-bearing nude mice, and the results showed that the survival time of the tumor-bearing nude mice of the PBS group was significantly lower than that of the cell treatment groups.

### Example 9-2: Animal Experiments

In order to examine the function of killer CD8⁺ T cells in killing tumor cells in vivo after intervention with specific anti-TRABD2B antibody, nude mice implanted with solid tumor MGC-803 were selected as research objects for adoptive CD8⁺ T cell treatment experiments.

After exploring of the treatment dose of CD8⁺ T cells for the MGC-loaded tumor models, 2×10⁶ was selected as the final treatment dose, and an in vivo animal study was performed, in which intervention with TRABD2B antibody was involved.

As shown in FIG. 11B, FIG. 11BA shows changes in body weight of nude mice relative to the day of tumor bearing (day 0), and according to FIG. 11BA, the first and second treatments were performed on day 10 and day 16, respectively. The results showed that the body weight of nude mice treated with CD8⁺ T cells and CD8⁺ T cells co-incubated with TRABD2B antibody increased constantly. FIG. 11BB shows changes in tumor volume of nude mice relative to 4 days before treatment (day 0).

According to FIG. 11BB, two treatments were performed on day 4 and day 10, respectively. The results showed that, after treatment, a significant decrease was observed in tumor volume of nude mice treated with CD8⁺ T cells and CD8⁺ T cells co-incubated with TRABD2B antibody, while the PBS control group showed an increasing tumor volume of nude mice, which eventually maintained stable.

### Example 9-3: Animal Experiments

In order to examine the function of CD8⁺ T cells in killing tumor cells in vivo after intervention with bispecific anti-TRABD2A/2B antibody, nude mice implanted with solid tumor MGC-803 were selected as research objects for adoptive CD8⁺ T cell treatment experiments.

After exploring of the treatment dose of CD8⁺ T cells for the MGC-loaded tumor models, 2×10⁶ was selected as the final treatment dose, and the second in vivo animal study was performed, in which intervention with the TRABD2A/2B bispecific antibody was involved.

As shown in FIG. 11C, FIG. 11CA shows changes in body weight of nude mice relative to the day of tumor bearing (day 0), and according to FIG. 11CA, the first and second treatments were performed on day 10 and day 16, respectively. The results showed that the body weight of nude mice treated with CD8⁺ T cells and CD8⁺ T cells co-incubated with the TRABD2A/2B bispecific antibody increased constantly. FIG. 11CB shows changes in tumor volume of nude mice relative to 4 days before treatment (day 0). According to FIG. 11CB, two treatments were performed on day 4 and day 10, respectively. The results showed that, after treatment, a significant decrease was observed in tumor volume of nude mice treated with CD8⁺ T cells and CD8⁺ T cells co-incubated with the TRABD2A/2B bispecific antibody, while the PBS control group showed an increasing tumor volume of nude mice, which eventually maintained stable.

To sum up, the results suggest that CD8⁺ T cell therapy can inhibit tumor growth and prolong the survival time of tumor-bearing nude mice.

The foregoing is the preferred embodiment of the present disclosure, and the present disclosure is not intended to be limited to the content disclosed in the embodiment and the drawings. All equivalents and modifications obtained without departing from the spirit disclosed in the present disclosure shall fall within the protection scope of the present disclosure.

1. A monoclonal antibody, comprising: a heavy chain amino acid sequence and a light chain amino acid sequence set forth in SEQ ID NOs: 1 and 2, respectively; a heavy chain amino acid sequence and a light chain amino acid sequence set forth in SEQ ID NOs: 3 and 4, respectively; a heavy chain amino acid sequence and a light chain amino acid sequence set forth in SEQ ID NOs: 5 and 6, respectively; a heavy chain amino acid sequence and a light chain amino acid sequence set forth in SEQ ID NOs: 7 and 8, respectively; a heavy chain amino acid sequence and a light chain amino acid sequence set forth in SEQ ID NOs: 9 and 10, respectively; a heavy chain amino acid sequence and a light chain amino acid sequence set forth in SEQ ID NOs: 11 and 12, respectively; a heavy chain amino acid sequence and a light chain amino acid sequence set forth in SEQ ID NOs: 13 and 14, respectively; a heavy chain amino acid sequence and a light chain amino acid sequence set forth in SEQ ID NOs: 15 and 16, respectively; a heavy chain amino acid sequence and a light chain amino acid sequence set forth in SEQ ID NOs: 17 and 18, respectively; a heavy chain amino acid sequence and a light chain amino acid sequence set forth in SEQ ID NOs: 19 and 20, respectively; a heavy chain amino acid sequence and a light chain amino acid sequence set forth in SEQ ID NOs: 21 and 22, respectively; a heavy chain amino acid sequence and a light chain amino acid sequence set forth in SEQ ID NOs: 23 and 24, respectively; a heavy chain amino acid sequence and a light chain amino acid sequence set forth in SEQ ID NOs: 25 and 26, respectively; a heavy chain amino acid sequence and a light chain amino acid sequence set forth in SEQ ID NOs: 27 and 88, respectively; a heavy chain amino acid sequence and a light chain amino acid sequence set forth in SEQ ID NOs: 29 and 30, respectively; a heavy chain amino acid sequence and a light chain amino acid sequence set forth in SEQ ID NOs: 31 and 32, respectively; or a heavy chain amino acid sequence and a light chain amino acid sequence set forth in SEQ ID NOs: 33 and 34, respectively.

2. The monoclonal antibody according to claim 1, comprising: the heavy chain amino acid sequence and the light chain amino acid sequence set forth in SEQ ID NOs: 7 and 8, respectively; or the heavy chain amino acid sequence and the light chain amino acid sequence set forth in SEQ ID NOs: 13 and 14, respectively; or the heavy chain amino acid sequence and the light chain amino acid sequence set forth in SEQ ID NOs: 23 and 24, respectively; or the heavy chain amino acid sequence and the light chain amino acid sequence set forth in SEQ ID NOs: 27 and 28, respectively; or the heavy chain amino acid sequence and the light chain amino acid sequence set forth in SEQ ID NOs: 29 and 30, respectively.

3. A nucleotide sequence encoding the heavy chain amino acid sequence of the monoclonal antibody according to claim 1.

4. A nucleotide sequence encoding the light chain amino acid sequence of the monoclonal antibody according to claim 1.

5. A vector, comprising a nucleotide sequence of the monoclonal antibody according to claim 1.

6. A host cell, comprising a nucleotide sequence of the monoclonal antibody according to claim 1.

7. A method for preparing the monoclonal antibody according to claim 1, comprising the steps of culturing a host cell containing the monoclonal antibody under suitable conditions and isolating the monoclonal antibody from a cell culture.

8. A method of treating tumor, comprising inhibition of TRABD2A protein, TRABD2B protein or TRABD2A protein and TRABD2B protein.

9. A method of inhibiting immune checkpoint, comprising using the monoclonal antibody according to claim 1.

10. The method according to claim 9, comprising the use of the monoclonal antibody as a tumor detection reagent or an anti-tumor drug.

11-12. (canceled)

13. A TRABD2A protein-blocked T cell, TRABD2B protein-blocked T cell or TRABD2A protein and TRABD2B protein-blocked T cell.

14. A method for preparing the T cell according to claim 13, comprising the step of contacting a T cell with TRABD2A monoclonal antibody, TRABD2B monoclonal antibody or TRABD2A and TRABD2B bispecific antibody.
